(19) United States
(12) Patent Application Publication
(10) Pub. No.: US 2024/0374707 A1
(43) Pub. Date: Nov. 14, 2024

(54) EXPRESSION SYSTEM AND NUCLEIC ACID-BASED PHARMACEUTICAL COMPOSITION COMPRISING SAME

(71) Applicant: SML BIOPHARM CO, LTD., Gyeonggi-do (KR)

(72) Inventors: Jae Hwan NAM, Gyeonggi-do (KR); Hyung Joon PARK, Gyeonggi-do (KR); Jae Yong KIM, Incheon (KR); Yoo Jin BANG, Gyeonggi-do (KR)

(73) Assignee: SML BIOPHARM CO, LTD., Gyeonggi-do, GG (KR)

(21) Appl. No.: 18/291,589

(22) PCT Filed: Jul. 26, 2022
(86) PCT No.: PCT/KR2022/010978

§ 371 Date: Jan. 24, 2024

(30) Foreign Application Priority Data
Jul. 29, 2021 (KR) 10-2021-0100099
Dec. 13, 2021 (KR) 10-2021-0177976

(51) Int. Cl. A61K 39/145; A61K 39/00; A61K 39/12; A61P 37/04; C07K 14/005

(52) U.S. Cl. CPC A61K 39/145; A61K 39/12; A61P 37/04; C07K 14/005; A61K 2039/53; C12N 2760/12022; C12N 2760/12034; C12N 2760/12071; C12N 2760/16022; C12N 2760/16034; C12N 2760/16071

(57) ABSTRACT

A nucleic acid molecule including a translation control element having a translation initiation activity and a coding region operably linked to the translation control element and encoding an immunogen of an influenza virus or a severe fever with thrombocytopenia syndrome virus (SFTSV) or fragments thereof is disclosed. The nucleic acid molecule or an expression system where the nucleic acid molecule is inserted can be used in a pharmaceutical composition for treating or preventing influenza or SFTS, for example, that an mRNA vaccine or a gene therapy platform.

### CROSS-REFERENCE TO RELATED APPLICATIONS

This application claims the priority of Korean Patent Application No. 10-2021-0049272, filed in Republic of Korea on Jul. 29, 2021, and Korean Patent Application No. 10-2021-017977, filed in Republic of Korean on Dec. 13, 2021, each of which is incorporated herein by reference in its entirety.

### TECHNICAL FIELD

The present disclosure relates to an expression system, and more particularly, to an expression system for a viral immunogen and a nucleic acid-based pharmaceutical composition using the system.

### BACKGROUND ART

Various infectious diseases transmitted to humans have been well known before modern times, and those infectious diseases have been developed into serious pandemic at times. The Black Death, which was prevalent in medieval Europe, killed a significant portion of the European population. Such pandemic of infectious diseases has continued even in modern times. It is known that the Spanish Flu in the early 20^{th} century killed more people than those who died in combat in World War I. In addition, COVID-19, which originated in Wuhan, China in 2019, has been causing various mutations and been still causing a global pandemic.

Vaccines have been widely used as a strategy to prevent such infectious diseases. Traditionally manufacture vaccines are protein vaccines that attenuate or inactivate antigens of viruses or bacteria causing those infectious diseases. Vaccines manufactured using traditional methods have effectively protected humans, but they have limitations. In particular, it is difficult to develop effective vaccines against various infectious pathogens that can evade the adaptive immune response. Additionally, the major difficulty in developing most new vaccines is the need for rapid development and large-scale distribution of the vaccine rather than effectiveness. Accordingly, the development of the vaccines using nucleic acids has become a good alternative. Even during the COVID-19 pandemic that occurred in 2019, nucleic acid-based mRNA vaccines were quickly developed, and the mRNA vaccines were administered worldwide with emergency use approval in each country. Therefore, there is a need to develop nucleic acid-based vaccine platforms that can efficiently prevent various infectious diseases other than COVID-19.

### BRIEF SUMMARY

An object of the present disclosure is to provide a nucleic acid and a recombination expression vector that can efficiently express an immunogen proteins or peptides associated with an infectious disease, and a nucleic acid-based pharmaceutical composition using thereof.

In one aspect, the present disclosure provides a nucleic acid molecule comprises a translation control element derived from troponin T1 (TNNT1), and a coding region linked operatively to the translation control element, wherein the coding region includes a nucleotide encoding an immunogen of an influenza virus or a severe fever with thrombocytopenia syndrome virus (SFTSV) or fragments thereof.

The translation control element can comprise a first translation control element upstream of the coding region and a second translation control element downstream of the coding region.

As an example, the first translation control element can comprise a nucleotide of SEQ ID NO: 1 or a transcript thereof, and the second translation control element can comprise a nucleotide of SEQ ID NO: 2 or a transcript thereof.

In one embodiment, the immunogen of the influenza virus can comprise hemagglutinin of the influenza virus or fragments thereof.

The immunogen of the influenza virus can comprise an amino acid of SEQ ID NO: 3.

The nucleotide encoding the immunogen of the influenza virus or fragments thereof can comprise a nucleotide of SEQ ID NO: 4 or a transcript thereof.

In another embodiment, the immunogen of the severe fever with thrombocytopenia syndrome virus can comprise a glycoprotein N of the sever fever with thrombocytopenia syndrome virus or fragments thereof.

The immunogen of the severe fever with thrombocytopenia syndrome virus can comprise an amino acid selected from SEQ ID NO: 5, SEQ ID NO: 7 and SEQ ID NO: 9.

The nucleotide encoding the immunogen of the severe fever with thrombocytopenia syndrome virus or fragments thereof can comprise a nucleotide selected from SEQ ID NO: 6, SEQ ID NO: 8 and SEQ ID NO: 10 or transcripts thereof.

For example, the nucleic acid molecule can comprise an RNA type nucleic acid molecule.

The nucleic acid molecule can further comprise at least one of the following nucleotides of a transcription control element linked operatively to the coding region, and a polyadenylation signal sequence or a poly adenosine sequence downstream of the transcription control element.

In another aspect, the present disclosure provides a recombination expression vector comprising the nucleic acid molecule.

In another aspect, the present disclosure provides a pharmaceutical composition for treating or preventing influenza or a severe fever with thrombocytopenia syndrome comprising the nucleic acid molecule or of claim 1 or an expression construct in which the nucleic acid molecule is inserted.

For example, the pharmaceutical composition can be a vaccine composition.

Alternatively, the pharmaceutical composition can further comprise least one of an adjuvant, a nucleic acid stabilizer and a lipid nano particle.

In another aspect, the present disclosure provides a process of treating or preventing influenza or a severe fever with thrombocytopenia syndrome comprising administering pharmaceutically acceptable amount of the nucleic acid molecule or the expression construct where the nucleic acid molecule is inserted to a subject.

Proteins and/or peptides derived from influenza virus or sever fever with thrombocytopenia syndrome virus (SFTSV) can be efficiently expressed through the nucleic acid molecule and the expression system into which the nucleic acid molecule is inserted of the present disclosure.

The nucleic acid molecule and/or the expression construct of the present disclosure can be used as a nucleic acid-based pharmaceutical active material and can be applied as a pharmaceutical composition to treat and/or prevent influenza caused by influenza virus or severe fever with thrombocytopenia syndrome (SFTS). Th₁ immune response as well as Th₂ immune response in a body can be efficiently induced by administering the nucleic acid molecule and/or the expression construct.

Therefore, the nucleic acid molecule and/or the expression construct including the nucleic acid molecule in accordance with the present disclosure can be used as an active ingredient in a drug such as a nucleic acid-based vaccine that can efficiently prevent or treat influenza caused by influenza virus or SFTS.

### BRIEF DESCRIPTION OF THE DRAWINGS

FIG. 1 is a schematic diagram illustrating components of a nucleic acid molecule that can efficiently express proteins or peptides causing infectious diseases in accordance with one embodiment of the present disclosure.

Each of FIGS. 2 and 3 illustrates an ELISA assay result by diluting serially immunoglobulins produced in mice sera immunized by a nucleic acid molecule encoding Hemagglutinin (HA) in accordance with Examples of the present disclosure.

FIG. 4 illustrates an ELISA assay result by diluting serially immunoglobulins produced in sera of mice sera immunized by a nucleic acid molecule encoding HA in accordance with Examples of the present disclosure.

FIG. 5 illustrates dilution times of inhibiting hemagglutination in HA inhibition (HI) assay using mice sera immunized by a nucleic acid molecule encoding HA in accordance with Examples of the present disclosure.

FIG. 6 illustrates neutralization capability by measuring viral titer with Micro-neutralization assay using mice sera immunized by a nucleic acid molecule encoding HA in accordance with Examples of the present disclosure.

FIG. 7 illustrates an ELISPOT assay result measuring numbers of cells producing an antigen-specific IFN-gamma in mice sera immunized by a nucleic acid molecule encoding HA in accordance with Examples of the present disclosure.

Each of FIGS. 8 and 9 illustrates a Flow Cytometry result using FACS by measuring CD4 T-cells and CD8 T-cells producing antigen-specific IFN-gamma in mice sera immunized by a nucleic acid molecule encoding HA in accordance with Examples of the present disclosure.

FIG. 10 is a photograph illustrating western-blotting results to certify that a nucleic acid molecule encoding a glycoprotein N of SFTSV in accordance with Examples of the present disclosure.

Each of FIGS. 11 and 12 illustrates an ELISA assay result by diluting serially immunoglobulins produced in mice sera immunized by a nucleic acid molecule encoding the glycoprotein N of SFTSV in accordance with Examples of the present disclosure.

FIG. 13 illustrates an ELISPOT assay result measuring numbers of cells producing an antigen-specific IFN-gamma in mice sera immunized by a nucleic acid molecule encoding the glycoprotein N of SFTSV in accordance with Examples of the present disclosure.

FIG. 14 illustrates FRNT assay result measuring neutralization antibody titer in mice sera immunized by a nucleic acid molecule encoding the glycoprotein N in accordance with Examples of the present disclosure.

### DETAILED DESCRIPTION

### Definitions

As used herein, the term “amino acid” is used in the broadest sense and is intended to include naturally occurring L-amino acids or residues thereof. Amino acid includes not only D-amino acid but also chemically-modified amino acids, for example, amino acid analogs, naturally occurring amino acids that is not typically incorporated into proteins, for example, norleucine, and chemically-synthesized compounds with amino acid-like properties known to a relevant art. For example, phenylalanine Phe or proline Pro analogs or mimetics each of which permits conformational limitations as the same as natural phenylalanine Phe or proline Pro is included in the definition of amino acid. Such analogs and mimetics are referred as “functional equivalences” of amino acids herein.

For example, synthetic peptides by standard solid-phase synthesis technique are not limited to amino acids encoded by corresponding genes, and allows the given amino acids to be substituted with much widely various ranges. Amino acids that are not encoded by the genetic code are referred as “amino acid analog” herein. For example, amino acid analog includes, but is not limited to, 2-amino adipic acid (Aad) to glutamic acid Glu and aspartic acid Asp; 2-amino pimelic acid (Apm) to Glu and Asp; 2-amino butyric acid (Abu) to methionine Met, leucine Leu and other aliphatic amino acids; 2-amino heptanoic acid (Ahe) to Met, Leu and other aliphatic amino acids; 2-amino iso-butyric acid (Aib) to glycine Gly; cyclohexyl alanine (Cha) to valine Val, Leu and isoleucine Ile; homo arginine (Har) to arginine Arg and lysine Lys; 2,3-diamino propionic acid (Dap) to Arg and histidine His; N-ethyl glycine (EtGly) to Gly, proline Pro and alanine Ala; N-ethyl asparagine (EtAsn) to asparagine Asn and glutamine Gln; hydroxyl lysine (Hyl) to Lys; allo hydroxyl lysine (AHyl) to Lys; 3-(and 4-) hydroxyl proline (3Hyp, 4Hyp) to Pro, serine Ser and threonine Thr; allo-isoleucine (AIle) to Ile, Leu and valine Val; 4-amidino phenyl alanine to Arg; N-methyl glycine (MeGly, sarcosine) to Gly, Pro and Ala; N-methyl isoleucine (MeIle) to Ile; norvaline (Nva) to Met and other aliphatic amino acids; ornithine (Orn) to Lys, Arg and histidine His; citrulline (Cit) and methionine sulfoxide (MSO) to Thr, Asn and Gln; and N-methyl phenyl alanine (MePhe), trimethyl phenyl alanine, halo-(F-, Cl-, Br- or I-) phenyl alanine or trifluoryl phenyl alanine to Phe.

As used herein, the term ‘peptide’ includes any of proteins, fragments of the proteins and peptides that are isolated from naturally-occurring environment or synthesized by recombinant technique or chemical synthesis. For example, the peptides of the present disclosure may comprise, but is not limited to, at least 5, for example, at least 10 amino acids.

In an exemplary embodiment, compound variants, for example, peptide variants substituted with one or more amino acids are provided. As used herein, the term “peptide variants” includes modified peptides that have one or more substitutions, deletions, addition and/or insertions of amino acids and exhibit substantially the same biological functions as the original peptide. The peptide variants should have an identity of 70% or more, preferably 90% of more, more preferably 95% or more as the original peptide.

Such amino acid substituents may comprise, but are not limited to, “Conservative” amino acid substituents. Alternatively, the amino acid substituents may include non-conservative variants. In one exemplary embodiment, the polypeptide variants may have amino acid sequences different from an original amino acid sequence by substitutions, deletions, additions and/or insertions of 5 or less amino acids. Besides, peptide variants may be changed by deletions or additions of amino acids that have minimal effects upon immunogenicity, a secondary structure, and hydropathic nature of a peptide.

As used herein, the term “conservative” substitution means that there are little changes in the secondary structure and hydropathic nature of polypeptides in case amino acids of the polypeptides changed to other amino acids. Such amino acid variations may be obtained based upon relative similarity of side chain substituents of amino acids, for example, polarity, charge, solubility, hydrophobicity, hydrophilicity, and/or amphipathic nature.

For example, amino acids may be classified to 1) hydrophobic (methionine, alanine, valine, leucine, isoleucine), 2) neutral hydrophilic (cysteine, serine, threonine, asparagine, glutamine), 3) acidic (aspartic acid, glutamic acid), 4) basic (histidine, lysine, arginine), 5) residues having influence on the chain directions (glycine, proline), and 6) aromatic (tryptophan, tyrosine, phenylalanine) based upon the common side chains properties. Conservative variation will accompany an exchange of one member in each of the classes for another member in the same class.

It has been known that any of arginine, lysine and histidine has positively charged residue; alanine, glycine and serine has similar sizes; phenylalanine, tryptophan and tyrosine has similar shapes by analyzing the size, shapes and kinds the amino acids side chain substituents. Accordingly, each of arginine, lysine and histidine; each of alanine, glycine and serine; and each of phenylalanine, tryptophan and tyrosine may be biologically functional equivalents by analysis with regard to the sizes, shapes and kinds of the amino acid side chain substituents.

Hydropathic index may be considered in introducing variations. Each amino acid is given hydropathic index based upon its own hydrophobicity and charge: Isoleucine (+4.5); Valine (+4.2); Leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

Hydropathic index of amino acids is very important in bestowing peptides or proteins with interactive biological functions. It has been known that similar biological activities may be maintained in only substituting amino acids with other amino acids having similar hydropathic indices. In case of introducing variations considering the hydropathic index, reciprocal substitutions among amino acids having hydropathic index value differences within preferably ±2, more preferably ±1, further more preferably ±0.5 are done.

Also, it is well known that reciprocal substitutions among amino acids having similar hydrophilicity induce proteins having equivalently biological activities. As disclosed in U.S. Pat. No. 4,554,101, following hydrophilicity value are accorded to each amino acid residue: Arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In case of introducing variations considering the hydrophilicity value, reciprocal substitutions among amino acids having hydrophilicity value differences within preferably ±2, more preferably ±1, further more preferably ±0.5 are done.

Amino acid exchanges in proteins that do not generally modify the molecular activities are known in the art (See, H. Neurath, R. L. Hill, The proteins, Academic Press, New York, 1979). The most commonly occurred exchanges are inter-exchange of amino acid residues between Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly.

Generally, the peptides (including fusion proteins) and polynucleotides described herein may be isolated. “Isolated” peptides or polynucleotides are separated from the original environment. For example, naturally occurring proteins are isolated by removing whole or part of co-existent material in a natural state. Such polypeptides should have purity of 90% or more, preferably 95% or more, more preferably 99% or more. Polynucleotides are isolated by cloning in the vectors.

As used herein, the term “polynucleotide” or “nucleic acid” are used inter-changeably, refers to polymers of any lengths of nucleotides, and includes comprehensibly DNA (i.e. cDNA) and RAN molecules. “Nucleotide”, which is a subunit of nucleic acid molecules, may comprises, but are not limited to, a deoxyribonucleotide, a ribonucleotide, a modified deoxyribonucleotide or a ribonucleotide, analogs thereof, and/or any substrates that can be incorporated into polynucleotides by DNA or RNA polymerase or synthetic reactions. Polynucleotide may comprise modified nucleotides, analogues having modified bases and/or polysaccharides such as methylated nucleotides and analogues thereof.

For example, the nucleotide can comprise 5-modified cytidine and/or 5-modified uridine. The 5-modified cytidine can include 5-halocyditine (e.g., 5-iodocytidine, 5-bromocytidine), 5-alkinylcytidine and/or 5-heterocyclylcytidine. 5-modified uridine can include 5-halouridin (e.g., 5-iodouridine or 5-bromouridine), 5-alkinyluridine and/or 5-heterocyclyl uridine. Alternatively or additionally, the 5-modified uridine can be a nucleotide containing 2-deoxyribose.

Some variations in nucleotides do not result in variations of peptides or proteins. Such nucleic acid variants may include any nucleic acid molecules having codons encoding functionally equivalent or identical amino acids (for example, 6 codons encodes Arg or Ser by the degeneracy of codons) or encoding biologically equivalent amino acids. On the other hand, other variations in nucleotides may induce changes in peptides or proteins. In spite of variations causing changes of amino acids of proteins, it is possible to obtain variant proteins that show substantially the same activities as the proteins of the present disclosure.

A person having ordinary skill in the art will appreciate that peptides and nucleic acids herein is not limited to the peptides and the nucleic acids described in the Sequence Listing. Rather, it is intended that peptides or the proteins as well as the nucleic acid molecules encoding the peptides or the proteins of the present disclosure may comprise any amino acid sequences or nucleotide sequences that has substantially the same biological functions such as vaccine and/or adjuvant. For example, the coding region operatively linked to the expression control element and/or biologically equivalents thereof that can be included in the recombination proteins/peptides expressed by the coding region can be any polynucleotide with varying base sequences and/or proteins/peptides with varying amino acid sequences exhibiting equivalent biological functions.

Considering the variations having the above biologically equivalent activities, the nucleic acid molecules encoding the peptide and/or the protein in accordance with the present disclosure can be interpreted to comprise any sequences showing substantial identity as the sequences described in the Sequence Listing. The substantial identity can mean any sequences having at least 61% homology, preferably at least 70% homology, more preferably at least 80% homology, and most preferably at least 90% homology in case analyzing the sequences aligned by using algorithms conventionally used in the art. The alignment process for comparing sequences is known to in the art.

As used herein, the term “vector” means a construct or a vehicle that can be transfected or delivered into the host cells, and enables one or more genes of interest (or target genes of target sequences) to be expressed within the cells. Additionally, a particular vector can instruct expressions of any genes of ORF (open reading frame) operatively linked to the vector. Such a vector can be referred to as “recombination expression vector” (or, “recombination vector”) herein.

As used herein, the term “expression control/regulation sequence” or “expression control/regulation element” may mean nucleic acid sequences regulating or controlling transcriptional processes of the nucleic acid molecules and/or translational processes of the nucleic acid molecules of transcripts. As used herein, the term “transcription control/regulation sequence” or “transcription control/regulation element” means that nucleic acid sequences regulating or controlling the transcriptional process of the nucleic acid molecules. For example, the transcription control sequence comprises promoters such as a constitutive promoter or an inducible promoter, enhancers, and the likes.

As used herein, the term “translation control/regulation sequence” or “translation control/regulation element” may be used to indicate nucleic acid sequences regulating or controlling the translational processes of the nucleic acid molecules of transcripts to the proteins or peptides. Each of the expression control sequence/element, the transcription control sequence/element and the translation control sequence/element is operatively linked to the target sequences to be expressed, for example, to be transcribed or translated.

As used herein, the term “operatively linked” means a functional linkage between expression control sequence (e.g., promoters, signal sequences, ribosome linkage sites, transcription termination sequence, and the likes) so that the expression control sequence may regulate transcriptions and/or translations of the other nucleic acid sequences.

### Nucleic Acid Molecule

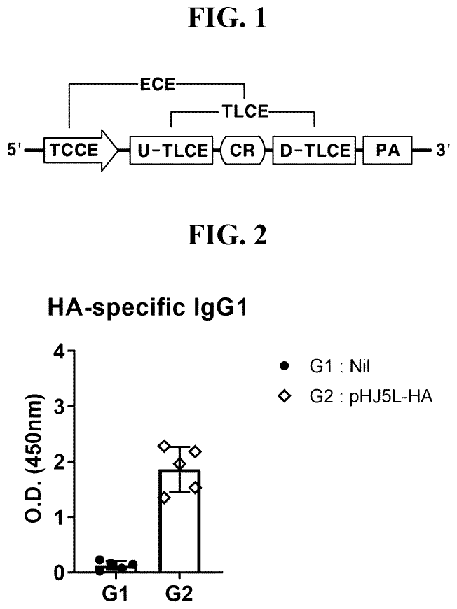

The present disclosure relates to a nucleic acid molecule that improves expression efficiency of an immunogenic target sequence linked operatively to an expression control sequence, and a nucleic acid-based vaccine using such an expression system. FIG. 1 is a schematic diagram illustrating components of a nucleic acid molecule that can efficiently express proteins or peptides causing infectious diseases in accordance with one embodiment of the present disclosure.

As illustrated in FIG. 1, the nucleic acid molecule can comprise an expression control element ECE, and a coding region CR linked operatively to the expression control element ECE and including an open reading frame (ORF) encoding an immunogen derived from viruses.

The expression control element ECE can include a translation control element TLCE derived from troponin 1 (TNNT1, slow skeletal muscle). The coding region CR can include an ORF linked operatively to the transcription control element TLCE and encoding an immunogen derived from an influenza virus and/or a severe fever with thrombocytopenia syndrome virus (SFTSV).

In one embodiment, the expression control element ECE can further include a transcription element TCCE operatively linked to the coding region CR and located upstream of the translation control element TLCE. In addition, the nucleic acid molecule can further at least one nucleotide of a polyadenylation signal sequence of a poly adenosine sequence PA located downstream of the expression control sequence ECE, for example, downstream of the translation control element TLCE.

The translation control element TLCE can include a nucleotide derived from TNNT1 linked operatively to the condign region CR inserted into the nucleic acid molecule as an ORF. For example, the translation control element TLCE can include a first translation control element U-TLCE located upstream of the coding region CR and/or a second translation control element D-TLCE located downstream of the coding region CR.

As an example, the first translation control element U-TLCE can include an entire 5′-untranslated region (5′-UTR) or a portion thereof having a cap-dependent translation initiation activity, and the second translation control element D-TLCE can include an entire 3′-untranslated region (3′-UTR) of a portion thereof corresponding the first translation control element U-TLCE.

Most eukaryotic mRNAs have 7-methyl-guanosine as a cap at 5′ terminus thereof. Translation initiation complex including various proteins recognizes the cap located at 5′ terminus and proceeds to AUG of an initiation codon to initiate protein synthesis. The cap structure located at 5′ terminus of the mRNAs enables the protein synthesis to initiate and to block destruction of mRNA structure from nuclease activity.

The first translation process in vitro can be performed by treating a plasmid DNA (pDNA) with restriction enzyme to linearize the pDNA, and attaching m7G(5′)-ppp(5′)G (referred to regular cap analog) to mRNA prepared using an appropriate RNA polymerase to create capped mRNA. Optionally, there is a method of performing in vitro transcription without the cap analog and then performing a cap reaction using a commercially available vaccinia virus capping enzyme. In this case, an ‘anti-reverse’ cap analog (ARCA) that prevents the reverse action of the cap can be used. When the ARCA is introduced, only 3′-O-methylation of methylated guanosine can bind to the nucleotide of unmethylated guanosine.

For example, each of the first translation control element U-TLCE and/or the second translation control element D-TLCE can include a nucleotide a cap-dependent translation initiation activity derived from an animal, for example, a mammal, more particularly a primates, and more particularly a human. As an example, the first translation control element U-TLCE can includes a nucleotide of SEQ ID NO: 1, which can be a portion of 5′-UTR derived from human TNNT1, or a transcript thereof, and the second translation control element D-TLCE can include a nucleotide of SEQ ID NO: 2, which can be a portion of 3′-UTR derived from TNNT1, or a transcript thereof, but is not limited thereto.

As an example, the first translation control element U-TLCE can be an element where the translation initiation complex can bind during the transitions of the peptide and/or the protein that can be expressed from the coding region CR. The first translation control element U-TLCE can be a cis-acting element inducing the translation of the target sequences in the coding region CR.

In one embodiment, when the first translation control element U-TLCE is an entire nucleotide having the cap-dependent translation initiation activity or a portion thereof, the nucleic acid molecule can include the second translation control element D-TLCE that can be located at a downstream of the coding region CR so that the expression efficiency of the target gene encoding the entire immunogen, which can be inserted into the coding region CR, derived from viruses or the fragments of the immunogen can be improved. The first translation control element U-TLEC and/or the second translation control element D-TLEC act important role of improving translation efficiency of the ORF or the transcript thereof encoding the peptide that can function as an immunogen in the influenza virus and/or SFTSV forming the coding region CR, and maintaining stably the mRNA of the transcript without decaying in the cell.

As an example, the first translation control element U-TLEC can be located at upstream of the coding region CR, and the second translation control element D-TLEC can be located at downstream of the coding region. In other words, the coding region can be inserted between the first translation control element U-TLEC and the second translation control element D-TLEC, and can include the target sequence encoding the peptide derived from the influenza virus and/or SFTSV, for example, infectious immunogens.

The influenza virus is a negative-sense virus and there are four kinds of influenza viruses, A, B, C and D among which types A and B are the ones that mainly causes illness in humans. Type A influenza virus is distinguished by the combination of the viral antigens hemagglutinin (HA) and neuramindase (NA). 3 to 5 million seasonal influenza patients show sever symptoms and 290,000 to 650,000 people among them die every year worldwide. There is no effective treatment to the influenza yet, and the best way to prevent it is with a vaccine. The inactivated influenza vaccine was first approved in the United States in 1945, and influenza vaccines are currently being developed through various methods using protein vaccines and virus-like particles (VLPs).

In one embodiment, the influenza virus immunogen being encoded in the coding region CR can include a hemagglutinin (HA) of the influenza virus or fragments thereof. For example, the hemagglutinin (HA) of the influenza virus immunogen can include an amino acid of SEQ ID NO: 3. As an example, the nucleotide encoding the influenza virus immunogen or fragments thereof can include, but is not limited to, a nucleotide of SEQ ID NO: 4 or a transcript thereof.

Sever fever with thrombocytopenia syndrome (SFTS) is a disease caused by SFTSV. The first patent with SFTS was reported in Republic of Korean and Japan in 2013. In Korea, the morality rate among 605 cases from 2013 to 2017 reached an average 20.9%. Symptoms of infection with SFTSV include high fever, fatigue, headache, muscle pain, vomiting and diarrhea, and in severe cases, decrease of plate count, and multiple dysfunctions of various organs including the kidney, liver and heart can be caused, and can be reached to death.

SFTS is most often caused by infections by ticks carrying the severe fever with thrombocytopenia syndrome virus (SFTSV), and occurs frequently in agricultural or forestry workers who spend a lot of time outdoors and often come into contact with grass where ticks mainly live. In Korea, may infections have been reported in the harvest season, between July and October. Although SFTS patients are infected by ticks, they can also be transmitted to other animal hosts such as sheep, pigs, dogs, cats, and the likes. Because the viral concentration in the blood of the infected patient is very high, it has been known that person-to-person transmission through blood is possible. In particular, as ticks migrate to North America, there is a risk that the disease could spread worldwide beyond Far East Asia. Accordingly, the World Health Organization (WHO) included SFTSV in the list of pathogens requiring attention in 2018.

SFTSV belongs to Bunyaviridae family and is a negative-sense RNA virus containing three segments. The genome of SFTSV is divided into an L segment, an M segment and an S segment. The L segment consists of 6385 nucleotides and contains an RNA-dependent RNA polymerase gene. The M segment consists of 3378 nucleotides and contains a glycoprotein N (Gn) and glycoprotein C (Gc) genes presented in an envelope. The S segment consists of 1744 nucleotides and encodes nucleoproteins and non-structural proteins.

Commercial treatments and vaccines for SFTSV have not been developed to date. The reason why the development is being delayed is that SFTS is a new infectious disease that was identified recently and that the number of patients was concentrated in Northeast Asia and the number of patent was not large, and there was not much global interest. Additionally, the lack of appropriate animal modes is one of the reasons why treatments and vaccines for SFTS were not developed.

As an example, the immunogen of the severe fever with thrombocytopenia syndrome virus (SFTSV) encoded in the coding region CR can include the glycoprotein N (Gn) or fragments thereof.

In one embodiment, the glycoprotein N (Gn) of the immunogen of the SFTSV can include a variant peptide thereof. For example, the variant peptide can have, but is not limited to, an amino acid selected from SEQ ID NO: 5 (herein referred to ‘Gn”), SEQ ID NO: 7 (herein referred to “GnΔTM”) and SEQ ID NO: 9 (herein referred to “GnΔSTEM”). As an example, the nucleotide encoding the SFTSV immunogen or fragments thereof can include, but is not limited to, a nucleotide selected from SEQ ID NO: 6, SEQ ID NO: 8 and SEQ ID NO: 10, or transcripts thereof.

For example, the nucleic acid molecule is an RNA type, the coding region CR can include a transcript having an ORF encoding the immunogen of the influenza virus (e.g., HA) and/or the immunogen of the SFTSV (e.g., glycoprotein), or fragments thereof.

There is no limitation in the length of the ORFs in the coding region CR, and the expression efficiency depending on the ORF length is not considered in developing a nucleic acid molecule, a recombinant vector, and pharmaceutical or medicinal applications for preventing or treating diseases using the molecule. Codon usage is not considered in developing human vaccines or gene therapies because codon usage basis in human has not influences on common peptides/proteins expression significantly.

But, it may be preferable that initial codons have Kozak sequence and nucleotides adjacent to termination codon may be optimized. If necessary, the third codon among genes or their transcript mRNA codon to be expressed may be changed “G/C” without changing amino acid so that mRNA may have improved stability.

Alternatively or additionally, other nucleotides can be inserted into the nucleic acid molecule so that the expression efficiency of the coding region CR as the ORF can be further increased. As an example, the nucleic acid molecule can have a transcription control element TCCE promoting the transcription of the target sequence adjacently to the first translation control element U-TLCE. For example, the transcription control element TCCE can be located upstream of the first translation control element U-TLCE. Such a transcription control element TCCE cannot be limited to a particular element.

In one exemplary embodiment, the transcription control element TCCE can be a promoter that promotes the transcriptions of the HA derived from the influenza virus or the Gn derived from SFTSV encoded in the coding region as the ORF form. The transcription control element TCCE acts on the animal cells such as the mammal cells so that the transcription control element TCCE can regulate the transcription of the immunogen derived viruses encoded in the coding region CR.

As an example, the transcription control element TCCE can include a mammal virus derived promoter, a mammal cell genome derived promoter, and/or bacteriophage derived promoter. For example, the transcription control element TCCE can include, but is not limited to, a Cytomegalovirus (CMV) promoter, an adenovirus late promoter, a vaccinia virus 7.5K promoter, an SV40 promoter, a tK promoter of HSV, a T7 bacteriophage promoter, a T3 bacteriophage promoter, an SM6 promoter, an RSV promoter, an EF1 alpha promoter, a metallothionein promoter, a beta-actin promoter, a human IL-2 gene promoter, a human IFN gene promoter, a human IL-4 gene promoter, a human lymphotoxin gene promoter, a human GM-CSF gene promoter, a tumor-cell specific promoter (e.g., a TERT promoter, a PSA promoter, a PSMA promoter, a CEA promoter, an E2F promoter and an AFT promoter) and a tissue-specific promoter (e.g., an albumin promoter).

In one embodiment, any transcription control element TCCE that can transcribe into mRNA from linearized DNA such as the T7 bacteriophage promoter, the T3 bacteriophage promoter, the SP6 bacteriophage promoter, and the likes can be located adjacently to the first translation control element U-TLCE, in particular, downstream of the first translation control element U-TLCE.

Additionally, any nucleotide that can induce the expression the ORF, which can be inserted into the coding region and can comprise the genes or transcripts thereof encoding the virus derived immunogenic peptides, can be inserted into the nucleic acid molecule other than the above translation control element TLCE, the coding region and the translation control element TCCE. In one embodiment, the nucleic acid molecule can include Kozak sequence that can be inserted between the transcription control element TCCE or the first translation control element U-TLCE, and the coding region CR.

Alternatively or additionally, the nucleic acid molecule can further include a polyadenylation signal sequence and/or a poly adenosine sequence PA downstream of the coding region CR, in particular, downstream of the second translation control element D-TLCE. The polyadenylation signal sequence and/or the poly adenosine sequence PA can stabilize the transcribed nucleic acid molecule and can further improve the translation efficiency of the ORF that comprise the genes or transcripts thereof encoding the virus derived immunogen peptides present in the coding region CR.

For example, when the nucleic acid molecule of the present disclosure is present as a transcript of mRNA, the poly adenosine sequence PA can be nucleotides of about 25 to about 400, for example, about 30 to about 400, about 50 to about 250 or about 60 to 250 adenosines.

In another embodiment, the nucleic acid molecule of the present disclosure is present as a DNA type, the polyadenylation signal sequence PA can be located downstream of the coding region CR. As an example, the polyadenylation signal sequence PA can be derived, but is not limited to, SV40, human growth factor (hGH), bovine growth hormone (BGH), rabbit beta-globin (rbGlob), and the likes.

Alternatively or additionally, the polyadenylation signal sequence or the poly adenosine sequence PA can include another signal sequences or linker sequences such as 5′-GATCATCAGT′-3′ between two nucleotides that each of which comprises multiple adenosines, for example, about 25 to about 400, about 30 to about 400, about 50 to about 250 or about 60 to about 250 or transcripts thereof.

The nucleic acid molecule can comprise at least one Cloning Site, preferably Multiple Cloning Site (MCS) for inserting the coding region CR therein. The at least one Cloning Site can comprise at least one restriction endonuclease recognition site and/or site cut by at least one restriction endonuclease. In one embodiment, the restriction endonuclease may comprise artificially engineered restriction endonuclease (e.g. zinc finger nuclease or restriction endonuclease based on DNA binding site of TAL effector or PNA-based PNAzymes) as well as naturally-occurring endonuclease found in bacterial or archaebacteria.

For example, the naturally-occurring restriction endonuclease may be classified into 1) Type I endonuclease (cuts sites spaced apart from recognition site and requires ATP, S-adenosyl-L-methionine and Mg²⁺), 2) Type II endonuclease (cuts specific sites within or spaced apart from recognition site and most requires Mg²⁺), 3) Type III endonuclease (cuts specific site spaced apart from recognition site and requires only ATP without hydrolysis of ATP), 4) Type IV endonuclease (targets modified sites such as methylation, hydroxyl methylation or glucosyl-hydroxyl methylation), and 5) Type V endonuclease (e.g. CRISPR cas9-mRNA complex), and the likes.

For example, the site recognized and/or cut by the following restriction endonuclease can be included, but is not limited to, in the Multiple Cloning Site: AngI, AatI, AbaI, BamH), BbvI, BcgI, BplI, BsmAI, Alw26I, BsrI, ClaI, EarI, Eco57I, EcoRI, EcoRII, EcoRV, FokI, HaeIII, HindIII, HpaIII, HphI, KpnI, MboI, MluI, NaeI, NdeII, NgoMIV, NlaIII, NotI, PacI, PstI, SacI, SacII, SalI, SfaNI, SmaI, TaqI, XbaI, XhoI and combinations thereof. In one embodiment, the cloning site can include, but is not limited to, at least one restriction endonuclease recognition site and/or restriction endonuclease cut site of SEQ ID NO: 16 to SEQ ID NO: 21.

The nucleic acid molecule can have a DNA type or an RNA type. In one embodiment, the nucleic acid molecule of the present disclosure can have the RNA type. When the nucleic acid molecule has the RNA type, for example, when the coding region CR comprises the ORF transcript encoding the immunogens of the influenza virus and/or the SFTSV, the RNA type nucleic acid molecule can have advantages over the DNA type nucleic acid molecule.

The RNA type nucleic acid molecule does not need to enter the nucleus of the host cells for transcription into mRNA, unlike the DNA type nucleic acid molecule. It is not possible for the RNA type nucleic acid molecule to be integrated into the host chromosome within the nucleus. Anti-biotic resistance genes, which are selection markers used to selectively produce the nucleic acid molecule in host cells, are unnecessary for the production of the RNA type nucleic acid molecule. The RNA does not induce long-time persistence genetic transformation because the RNA has a shorter half-life than DNA.

The RNA type nucleic acid molecules can induce a desired in vivo immune response even when used in relatively small amounts compared to the DNA type nucleic acid molecules. Additionally, the entire manufacturing processes can be artificially controlled in preparing the RNA type nucleic acid molecules, so that the RNA type nucleic acid molecules can be safely produced in small-scale GMP (good manufacturing practice) production facilities without the risk of biological contamination. There is no need to directly treat the infectious agents in producing the RNA nucleic acid molecules. Instead, only the nucleic acid sequences of the neutralizing antibody-inducing part (neutralizing epitope) of the infectious agent to be expressed is artificially synthesized, and performed in vitro transcription (IVT) in mass producing the mRNA. Recently, reagents related to IVT reactions, particularly, DNA-dependent RNA polymerase have been improved, making it possible to rapidly produce large amounts of RNA within 1 to 2 weeks using a small amount of DNA template.

The RNA type RNA nucleic acid molecules can induce a stronger immune response than naked DNA nucleic acid molecules. The RNA type nucleic acid molecules itself can produce a complex antigen within the cell, which can function as an ideal adjuvant by accessing MHC (Major histocompatibility complex) class II of antigen-presenting cells. Additionally, multiple antigens to induce immune responses can be produced simultaneously, mixed, and then immunized. There are no special restrictions on the gene length of the antigen to be expressed, which can increase the applicability and simplicity of producing the mRNA type nucleic acid molecules.

Appropriate transcription control element TCCE to enable the IVT can be located upstream of the first translation control element U-TLCE in case utilizing the RNA type nucleic acid molecule. Since the RNA type nucleic acid molecule can be synthesized through the IVT process, there is no need directly deal with live viruses or pathogenic micro-organisms used in the production of general attenuate or killed vaccines, and there is no need to culture host cells such as yeast, E. coli, or insect cells, which should be used to produce recombinant proteins/peptides.

When the DNA type nucleic acid molecules inserted into a vector are transcribed into mRNA type through the IVT process, the RNA type nucleic acid molecules can be synthesized in vitro using the DNA as a template whose termini have been linearized by restriction endonuclease and the RNA polymerase. The transcription control element TCCE such as a promoter element, for example, derived from bacteriophages, can be located upstream of the translation control element TLCE so that the linearized DNA can be transcribed into the RNA type.

### Recombination Expression Vector, Expression Construct and Nucleic Acid Molecule Injection

The nucleic acid molecule illustrated in FIG. 1 can be inserted into a recombination expression vector. The recombination expression vector can include the translation control element TLCE having the translation initiation activity, and the coding region. The recombination expression vector can further include the transcription control element TCCE and/or the polyadenylation signal sequence and the poly adenosine sequence PA. In other words, the recombination expression vector can include the nucleic acid molecule described referring to FIG. 1. The nucleic acid molecule can join other nucleic acids to encode fusion proteins or fusion peptides. The nucleic acid molecule can be injected to a body as an expression construct of a gene carrier.

Vectors that can be used as the gene carrier can be manufactured in various forms. The vectors can include viral vectors, DNA or RNA expression vectors, plasmids, cosmids or phage vectors, DNA or RNA expression vectors linked to CCA (cationic condensing agents) such as DNA or RNA vectors packaged liposomes or niosomes containing plasmids, and specific eukaryotic cells such as producer cells.

In one embodiment, the nucleic acid molecule of the present disclosure can be constructed to enter the mammal cells and to be expressed. Such construction can be particularly useful for using treatments and/or preventions of the infectious diseases. There is much methods for expressing nucleic acid molecules in the host cells and any appropriate methods can be used. For example, the nucleic acid molecule in accordance with the present disclosure can be inserted into any vectors constituting the expression construct of the gene carrier system.

A category of vectors is a ‘plasmid’ which refers to a circular, double-stranded DNA loop into which additional nucleic acid fragments can be ligated. Another category of vectors is a phage vector. Still another category of vectors is viral vectors into which additional nucleic acid fragments can be ligated into the viral genome. Specific vectors can replicate autonomously into the host cells having the transfected the vectors (e.g., viral vectors and episome mammalian vectors having bacterial replication origins). Other vectors (e.g., non-episome mammalian vectors) can be integrated into the genome of a host cell as they transfect the host cell, and thereby, being replicated together with the genome of the host cell. Generally, the expression vectors, which may be useful for recombinant DNA technologies, exist as a shape of plasmid.

As an example, the nucleic acid molecule can be inserted to the host cells using viral gene transfer system. The viral vector into which the nucleic acid molecule can be inserted can include vectors derived from an adenovirus, an adeno-associated virus (AAV), a retrovirus, a vaccinia or other fox viruses (e.g., avian pox virus), a lentivirus, a herpes simplex virus. For example, viral vectors can include, but is not limited to, lentivirus derived vectors such as a human immunodeficiency virus (HIV), a simian immunodeficiency virus (SIV); retrovirus viruses derived from murine retroviruses, a gibbon ape leukemia virus, adeno-associated viruses (AAVs) and adenovirus. In addition, retrovirus vectors derived from murine leukemia virus (MuLV), gibbon ape leukemia virus (GaLV), ecotropic retroviruses can be used. The vector system in accordance with the present disclosure can be constructed through various methods known in the art.

If necessary, the above nucleic acid molecule can be inserted to an appropriate vector and then can be modified to the RNA type nucleic acid molecule through the IVT process.

The techniques for inserting the nucleic acid molecule, for example DNAs into those vectors are well-known in the art. Additionally, the retrovirus vector can further include genes for a selectable marker that makes easily to certify or select the transfected cells and/or genes encoding a ligand acting as a receptor for the specific target cells. Targeting can be done through conventional methods using antibodies.

It is possible to use plural vectors that are commercially available and known to in the art for the purposes of the present disclosure. Selecting appropriate vectors will be mainly dependent upon the sizes of the nucleic acid molecules to be inserted into the vectors and specific host cells transfected with the vectors. Each vector contains various components, depending upon its functions (amplification and/or expression of foreign polynucleotides) and compatibilities to the specific host cells having thereof. Vector components generally comprises, but are not limited to, replication origins (especially if the vector is inserted into prokaryotes), selection marker genes, promoters, ribosome binding sites (RBS), signal sequences, foreign nucleic acids insert, and a transcription termination sequence.

For example, the expression vector of the present disclosure can comprise another expression control elements, which may have an influence on the expression of influenza virus or SFTSV derived immunogens, such as an initiation codon, a termination codon, a polyadenylation signal sequences, enhancers, signal sequences for membrane-targeting or secretions, and the likes, and encoded in the coding region CR. The enhancer sequences are nucleic acid sequences which are located at various sites with regard to transcription control sequence, e.g. promoter and increase transcription activity compared to a transcription activity by the promoter without the enhancer sequences.

The signal sequences can comprise PhoA signal sequence, OmpA signal sequence, and the likes in case the host cell is bacteria in Escherichia spp., α-amylase signal sequence, subtilisin sequence and the likes in case the host cell is bacteria in Bacillus spp., MF-α signal sequence, SUC2 signal sequence and the likes in case the hose cell is yeast, and insulin signal sequence, α-interferon signal sequence, antibody molecule signal sequence and the likes in case the host cell is mammals.

Specific vectors (e.g., viral vectors and episome mammalian vectors having bacterial replication origins) can replicate autonomously into the host cells having the transfected the vectors. Other vectors (e.g., non-episome mammalian vectors) can be integrated into the genome of a host cell as they transfect the host cell, and thereby, being replicated together with the genome of the host cell.

The vector system of the present disclosure can be constructed using various methods in the art. The vector of the present disclosure can be constructed as typically cloning purposes or expression purposes. The vector of the present disclosure can be constructed using prokaryotic cells or eukaryotic cells as hosts. For example, the vectors, which can be used in the present invention, can be manufactured by manipulating the usually used vectors in the art, plasmid (e.g., pSC101, ColE1, pBR322, pUC8/9, pHC79, pUC19, pET and the likes), phage (e.g., λgt4λB, λ-Charon, λΔz1, λGEM.TM.-11, M13 and the likes), virus (e.g., SV40) and the likes).

Constitutive or inducible promoters can be used in the present disclosure depending upon the necessity of specific environments that can be certified by a person having an ordinary skill in the art. Plural promoters that recognized by various possible host cells have been widely known in the art. Selected promoters can be linked operatively to the nucleic acid molecule having the coding region CR comprising the ORF of genes or transcripts encoding the immunogenic peptides or proteins by removing the promoters from supplier nucleic acid molecule through restriction endonuclease digestions and then inserting the isolated promoter sequences into the selection vectors. It is possible to direct amplification and/or expression of the genes or transcripts of the coding region CR using both natural promoter sequences and a plurality of foreign promoters. But, foreign promoters are generally more preferable to the natural targeting polypeptide promoters because the foreign promoters allows much transcription and high yield of the expressed target genes compared to the natural targeting polypeptide promoters.

For example, when the vector of the present disclosure is an expression vector and a host cell is eukaryotes, the vector can comprise the transcription control element TCCE such as strong promoters for proceeding with the transcription (e.g., a tac promoter, a lac promoter, a LacUV5 promoter, an lpp promoter, a pLλ promoter, a pRλ promoter, a rac5 promoter, an amp promoter, a recA promoter, a SP6 promoter, a trp promoter and a T7 promoter), the translation control element TLCE for initiating translation, and transcription/translation termination sequences. In case of using E. coli as the host cell, promoters and operator sites of E. coli tryptophan biosynthetic pathway (Yanofsky, C., J. Bacteriol., 158:1018-1024, 1984), and pLλ promoter can be used as the expression control site.

Alternatively, when the vector of the present disclosure is an expression vector and uses eukaryotes as the host cell, the vector can comprise promoters such as promoters derived from the genome of the mammalian cells (e.g., metallothionein promoter), promoters derived from mammalian viruses (e.g., adenovirus late promoter, vaccinia virus 7.5K promoter, SV40 promoter, cytomegalovirus promoter and tK promoter of HSV) or promoters derived from phages (e.g., T7 promoter, T3 promoter and SM6 promoter), and polyadenylation signal sequences as a transcription termination signal sequence.

Additionally, when the recombination vector of the present disclosure is a replicable expression vector, the recombination vector can comprise a replication origin, which is a specific nucleic acid sequence for initiating replication. In addition, the recombination vectors can comprise sequences encoding selectable markers. The selectable markers are intended to screen transfected cells by the vectors and markers giving selectable phenotypes such as drug resistances, nutritional requirements, cytotoxic agent resistances, or expressions of surface proteins may be used. The vectors of the present invention can comprise antibiotics resistant genes which have been conventionally used in the art, for example, ampicillin, gentamicin, carbenicillin, chloramphenicol, streptomycin, kanamycin, geneticin, neomycin, and tetracycline resistant genes as selectable markers. It is possible to screen the transfected cells because only cells expressing the selectable markers can survive in an environment of treating elective agents. Representative example of the selectable markers may comprise an auxotrophic marker, ura4, leu1, his3 and the likes, but the selectable markers can be used in the present invention is not limited to such an example.

Various in vitro amplification techniques that amplify sequences sub-cloned to expression vectors have been known. There are PCR (polymerase chain reaction), LCR (ligase chain reaction), Qβ-replicase amplification and techniques using other RNA polymerases in such techniques.

The vector of the present disclosure may be fused with other sequences in order to facilitate the purification of the recombinant protein or peptide expressed therefrom. Fused sequences comprises glutathione S-transferase (Pharmacia, USA), maltose binding protein (NEB, USA), FLAG (IBI, USA), 6× His (hexahistidine, Quiagen, USA) and the likes, and 6× His is most preferable. Owing to the additional sequences for purification, the proteins expressed in the host cell are purified with promptness and ease through affinity chromatography assay. If necessary, a sequence encoding Fc fragments may be fused with the vector in order to facilitate extracellular secretion of those peptides.

In accordance with an exemplary embodiment of the present disclosure, the fusion proteins expressed by the vector which comprises the fused sequences can be purified by affinity chromatography. For example, it is possible to purify the peptides or proteins of interest with promptness and with ease by using glutathione as a substrate of glutathione-S-transferase when glutathione-S-transferase is fused with the vector, and by using Ni-NTA His-binding resin column (Novagen, USA) when the vector comprises 6× his.

It is possible to use any host cells known in the art as long as the host cells make the vectors stably and continuously clone and express. For example, host cells may comprise E. coli JM109, E. coli BL21 (DE3), E. coli RR1, E. coli LE392, E. coli B, E. coli X 1776, E. coli W3110, Bacillus strains such as Bacillus subtilis, Bacillus thuringiensis, and Enterobacteriaceae strains such as Salmonella typhimurium, Serratia marcescens and various Pseudomonas species. Besides, yeast (Saccharromyce cerevisiae), insect cells (e.g., SF9 cell), human cells (e.g., CHO (Chinese hamster ovary) cell, W138, BHK, COS-7, 293, HepG2, 3T3, RIN and MDCK cell lines) may be used as host cells in case of transfecting eukaryotes with the vectors of the present disclosure.

The vector of the present disclosure can be used to modify genetically the cells in vivo, ex vivo or in vitro. The methods for modifying genetically the cells such as a technique of transfecting or transducing cell into viral vectors, a calcium phosphate precipitation method, a technique of fusing recipient cells with bacterial protoplast including DNAs, a technique of treating liposome or microsphere containing DNA with recipient cells, endocytosis method (e.g., DAEA dextran, receptor-mediated endocytosis), electroporation techniques and micro-injection techniques are known in the art.

For example, when the host cells are prokaryotes, the vectors can be injected into the host cells by CaCl₂ method, Hanahn method and/or electroporation method. When the host cells are eukaryotes, the vector can be injected into the host cells by a micro injection method, a calcium phosphate method, an electroporation method, a liposome-mediated transfection method, a DEAE-dextran treatment method, and/or a gene bombardment method. The vector injected into the host cells can be expressed within the cells in which large amount of recombinant peptides or proteins are obtained. For example, when the expression vector includes a lac promoter, it is possible to induce gene expression by treating IPTG to the host cells.

### Pharmaceutical Composition

In another aspect, the present disclosure relates to a pharmaceutical composition, for example, a vaccine composition, for treating or preventing influenza or SFTS, comprising the above nucleic acid molecule and/or an expression construct of an expression system, as an active ingredient, into which the above nucleic acid molecule is inserted. In other words, the nucleic acid molecule or the expression construct including the nucleic acid molecule acts as an immunogen and can be an active ingredient of the pharmaceutical composition for treating or preventing influenza or SFTS.

Accordingly, in accordance with another aspect, the pharmaceutical composition for treating or preventing influenza or SFTS comprising an effective amount of the nucleic acid molecule, and optionally a pharmaceutical carrier. In this case, the nucleic acid molecule can be administered to a subject directly. As used herein, the term “pharmaceutically effective amount” means an amount of sufficiently accomplishing efficacy or activation of the nucleic acid molecule of the present disclosure.

In accordance with another aspect, the present disclosure provides a pharmaceutical composition for treating influenza or SFTS comprising the expression construct into which the nucleic acid molecule is inserted, and optionally a pharmaceutical carrier. In another aspect, the present disclosure provides a method for preventing or treating influenza or SFTS comprising administering a pharmaceutically acceptable amount of the nucleic acid molecule or the expression construct.

The pharmaceutical composition comprising the pharmaceutically acceptable amount of the nucleic acid molecule or the expression construct comprises the carrier, a diluent and/or an excipient. In one embodiment, the nucleic acid molecule or the expression construct of the active ingredient can be formulated by mixing to a desired degree of purity with a physiologically acceptable carrier, i.e., a carrier non-toxic to recipients at the dosage and concentrations in herbal dosage forms, at ambient temperature, appropriate pH. The pH of the formulation largely depends on the particular applications and concentration of the compound, but preferably ranges from about 3 to about 8. In another embodiment, the compound is sterile. The compound can be stored, for example, as solid or amorphous composition, lyophilized preparations or aqueous solutions.

In one embodiment, the nucleic acid molecule or the expression construct can be stabilized in the pharmaceutical composition using a nucleic acid stabilize such as a cationic polymer, a cationic peptide or a cationic polypeptide. The cationic (poly) peptide that can be used as the nucleic acid stabilizer can include multiple cationic polymer such as poly-lysine or poly-arginine, a cationic lipid or lipofectant. For example, the stabilizer can include histone, nucleoline, protamine, oligo-fectamine, spermine or spermidine, and cationic polysaccharides, in particular chitosan, TDM, MDP, muramyl dipeptide, pluronics, and/or derivatives thereof. Histones and protamines are cationic proteins which naturally compact DNA.

As an example, histones that can form a complex with the nucleic acid molecule or the expression construct can include histones H1, H2a, H3 and H4. The protamines that can form a complex with the nucleic acid molecule may be made of protamine P1 or P2 or cationic partial sequences of protamine. If necessary, other compounds that can form a complex with the nucleic acid molecule may be another adjuvant additionally used. The additionally used adjuvant can improve the immunogenicity of the pharmaceutically active material, the nucleic acid molecule.

In one embodiment, the adjuvant can include protamine, nucleoline, spermine, spermidine, cationic polysaccharides, stabilized cationic peptide or polypeptide, in particular, chitosan, TDM, MDP, muramyl dipeptide, pluronics, alum solution, aluminum hydroxide, ADJUMER (polyphosphazene); aluminum phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE (propanediamine); BAY R005 ((N-(2-deoxy-2-L-leucylamino-b-D-glucopyranosyl)-N-octadecyldodecanoyl-amide hydroacetate); CALCITRIOL (1α,25-dihydroxy-vitamin D3); calcium phosphate gel; CAP™ (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of i) N-acetylglucosaminyl-(P1-4)-N-acetylmuramyl-L-alanyl-D-glutaminc (GMDP), ii) dimethyldioctadecylammonium chloride (DDA), iii) zinc-L-proline salt complex (ZnPro-8); GM-CSF); GMDP (N-acetylglucosaminyl-(b1-4)-N-acetylmuramyl-L-alanyl-D-isoglutamine); imiquimod (1-(2-methypropyl)-1H-imidazo[4,5-c]quinoline-4-amine); Imm Ther (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-1-beta; interleukin-2; interleukin-7; interleukin-12; ISCOMS (“Immuno-stimulating Complexes”); ISCOPREP 7.0.3.; liposomes; LOXORIBINE (7-allyl-8-oxoguanosine (guanine)); LT oral adjuvant (E. coli labile enterotoxin-protoxin); microspheres and microparticles of any composition; MF59™; (squalene-water emulsion); MONTANIDE ISA 51 (purified incomplete Freund's adjuvant); MONTANIDE ISA 720 (metabolisable oil adjuvant); MPL (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))ethylamide, monosodium salt); MURAMETIDE (Nac-Mur-L-Ala-D-Gln-OCH₃); MURAPALMITINE and D-MURAPALMITINE (Nac-Mur-L-Thr-D-isoGln-sn-glyceroldipalmitoyl); NAGO (neuraminidase-galactose oxidase); nanospheres or nanoparticles of any composition; NISVs (non-ionic surfactant vesicles); PLEURAN (beta-glucan); PLGA, PGA and PLA (homo- and co-polymers of lactic acid and glycolic acid; micro-/nano-spheres); PLURONIC L121; PMMA (polymethyl methacrylate); PODDS (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, Ala.); STIMULON (QS-21); Quil-A (Quil-A saponin); S-28463 (4-amino-otec-dimethyl-2-ethoxymethyl-1H-imidazo[4,5-c]quinoline-1-ethanol); SAF-1 (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid (N-acetylglucosaminyl-N-acetylmuramyl-L-Ala-D-isoGlu-L-Ala-dipalmitoxypropylamide); Theronyl-MDP (Termurtide or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and the likes

As an example, the adjuvant contained in the pharmaceutical composition can include, but is not limited to, alum (inducing Th₂ immune response and enhancing humoral immune response); oil-in-water emulsion type adjuvant (enhancing antibody-induced immune response and inducing Th₁ immune response in balance) such as MF59, AS03, AS04 (mixing MPL of TLR-4 agonist with alum; GSK), AddVax (squalane base; InvivoGen); LPS (lipid-polysaccharide), agonist of pattern recognition receptors (PRRs) such as Toll-like receptors (TLRs), RIG-I-like receptors (RLRs) and NOD-like receptors (NLRs); Poly: C; imidazoquinolines (imiauinod or R848), CpG oligonucleotides, and the likes.

In case of mixing the nucleic acid molecule or the expression construct with the adjuvant, the mixing ratio is not particularly limited. As an example, the nucleic acid molecule or the expression construct of the present disclosure and the adjuvant can be mixed with a weight ratio of 100:1 to 1:100, preferably 10:1 to 1:10, more preferably 5:1 to 1:5, and most preferably 3:1 to 1:3.

Optionally, the pharmaceutical composition can be prepared as a sustained-release formulation. Suitable sustained-release formulations include semi-permeable matrices of solid hydrophobic polymers containing the nucleic acid molecule or the gene carrier. The matrices can be in the form of molded articles, for example, films or micro-capsules. Examples of the sustained-release matrices can include polyesters, hydrogels (e.g., poly(2-hydroxylethyl-methacrylate) or poly (vinyl alcohol)), poly lactide, a copolymer of L-glutamic acid and gamma-ethyl-L-glutamate, non-degradable ethylene-vinyl acetate, a degradable lactic acid-glycol acid copolymer, and poly-D-(−)-3-hydroxybutyric acid.

Alternatively, the pharmaceutical composition can include a lipid nano particle (LNP) that can protect the nucleic acid molecule as the active ingredient and can improve a body injection activity. The lipid nano particle can plural lipid molecules that are physically associated to each other, and can include a microsphere (including mono-layered or multi-layered vesicles, for example, liposome), phase dispersed in an emulsion, micelle, and/or internal phase of suspensions. The lipid nano particle can be used to capsulate nucleic acid molecule of the present disclosure or the peptide expressed from the nucleic acid molecule.

The formulation containing the cationic lipid can be useful for delivering poly valent cations such as the nucleic acid molecule. Other lipid can be included in the pharmaceutical composition is a neutral lipid (i.e., uncharged or positively ionic lipid), a anionic lipid, a helper lipid enhancing transfection and stealth lipid that can increasing the time period for the nano particles in the body). Appropriate cationic lipids, neutral lipids, anionic lipids, helper lipids and stealth lipids are described in WO 2016/010840 A1 which is incorporated herein by reference.

For example, the lipid of the capsulation can be the cationic lipids and/or a biodegradable lipid. As an example, such a lipid can include (9Z-12Z)-3((4,4-bis(octyloxy)butanol)oxy)-2(((3-(diethylamino)propoxy)carbonyl)oxy)methyl)propyloctadeca-9,12-dienoate, ((5-((dimethylamino)methyl)-1,3-phenylen)bis(oxy))bis(octan-8,1-diyl)-bis(decanoate), 2-((4-(((3-(dimethylamino)propoxy)carbonyl)oxy)hexadecanoyl)oxy)propan-1,3-diyl(9Z,9′Z, 12Z,12′Z)-bis(octadeca-9,12-dienoate), 3-(((3-(dimethylamino)propoxy)carbonyl)oxy)-13-(octanoyloxy)tridecyl-3-octylundecanoate, heptatriaconta-6,9,28,31-tetraen-19-yl4-(dimethylamino)butanoate (known to Dlin-MC3-DMA (MC3)).

Suitable neutral lipid can include a neutral, uncharged or cationic lipid. The neutral lipid can include, but is not limited to, 5-heptadecylbenzene-1,3-diol (resorcinol), dipalmitoylphosphatidylcholine (DPPC), distearoylphosphatidylcholine (DSPC), phosphocholine (DOPC), dimyristoylphosphatidylcholine. (DMPC), phosphatidylcholine (PLPC), 1,2-distearoyl-sn-glycero-3-phosphocholine (DAPC), phosphatidylethanolamine (PE), egg phosphatidylcholine (EPC), dilauroylphosphatidylcholine (DLPC), 1-myristoyl-2-palmitoyl phosphatidylcholine (MPPC), 1-palmitoyl-2-myristoylphosphatidylcholine (PMPC), 1-palmitoyl-2-Stearoylphosphatidylcholine (PSPC), 1,2-diarachidoyl-sn-glycero-3-phosphocholine (DBPC), 1-stearoyl-2-palmitoyl phosphatidylcholine (SPPC), 1,2-dicicosenoyl-sn-glycero-3-phosphocholine (DEPC), palmitoyloleoylphosphatidylcholine (POPC), lysophosphatidylcholine, diolcoylphosphatidylethanolamine (DOPE), and the likes.

The helper lipid can enhance transfection and/or can enhance membrane fusogenicity. The helper lipid can include steroids, sterols, and alkyl resorcinols. For example, the helper lipid can include cholesterol, 5-heptadecyl resorcinol, and cholesterol hemi succinate.

The stealth lipid can assists the formulation process by reducing particle aggregations and controlling particle sizes, or regulate pharmacokinetic properties of the lipid nano particle (LNP). As an example, the stealth lipid can include a polymer with a hydrophilic head such as polyethylene glycol (PEG), polyethylene oxide (PEO), poly(oxazoline), poly(vinyl alcohol), poly(glycerol), poly(N-vinyl pyrrolidone), poly amino acid, and poly N-(2-hydroxypropyl)methacryl amide. For example, the stealth lipid can include, but is not limited to, PEG-dilauroyl glycerol, PEG-dimyristoyl glycerol (PEG-DMG), PEG-dipalmitoyl glycerol, PEG-distearoyl glycerol (PEG-DSG), PEG-dilauroyl glycamide, PEG-dimyristyl glycamide, PEG-cholesterol (1-[8′-cholest-5-en3[beta]-oxy]caboxamido-3′,6′-dioxaoctanyl)carbamoyl-[omega]-methyl-poly(ethylene glycol), PEG-DMB (3,4-ditetradecoxylbenzyl-[omega]-methyl-poly(ethylene glycol)ether), and the likes.

The nucleic acid molecule of the present disclosure can induce non-antigen specific immune responses. T lymphocytes is differentiated into T-helper 1 (Th₁) cells and T-helper 2 (Th₂) cells with regard to immune responses and immune system can destroy intra-cellular pathogens (e.g. antigens) by Th₁ cells and extra-cellular pathogens by Th₂ cells. Th₁ cells helps cell-mediated immune response by activating macrophages and cytotoxic T-cells, while Th₂ cells facilitates humoral immune responses by enhancing B-cell for transformation into cytoplasmic cells and by forming antibodies against the antigens. Accordingly, the ratio of Th₁ cells/Th₂ cells in immune response is very significant. The nucleic acid molecule of the present disclosure can enhance and induce Th₁ immune response, i.e., cell-mediated immune responses. Accordingly, when the nucleic acid molecule of the present disclosure is injected into body together with a pharmaceutically active ingredient, e.g., immunity enhancing components, the pharmaceutical composition can further enhance the specific immune response induced by the pharmaceutically active ingredient.

In one embodiment, the pharmaceutical composition can further include pharmaceutically active substance other than the nucleic acid molecule described above. The pharmaceutically active substance can be an immune-enhancing component. As an example, the pharmaceutically active substance can be any compound having treatment and/or prevention effect against cancers, infectious diseases, auto-immune diseases and allergies. For example, the pharmaceutically active substance can include peptides, proteins, nucleic acids, therapeutically active organic or inorganic low-molecular compounds, sugars, antigens or antibodies, drugs known in the art, antigen cells, fragments of antigen cells, cell debris, (for example, attenuated or inactivated) pathogens (viruses and bacteria) modified by chemicals or light radiation, and the likes.

As an example, the antigen of one of the pharmaceutically active substance can include peptides, polypeptides, proteins, cells, cell extracts, polysaccharides, complex saccharides, lipids, glycolipids and carbohydrates. For example, the antigen can have secreted forms of surface antigens of tumor cells, in particular, viral pathogens, bacterial pathogens, fungal pathogens or protozoan pathogens. For example, the antigen can be present in the nucleic acid molecule of the present disclosure, or can be present as a heptane combined to an appropriate carrier. Other antigens, inactivated or attenuated pathogens can be used.

The pharmaceutical composition can further include a pharmaceutically acceptable carrier in addition to the nucleic acid molecule and the pharmaceutically active substance. In one embodiment, the pharmaceutically acceptable carrier can include pyrogen-free water; isotonic saline or buffered (aqueous) solution such as phosphate or citrate; plant oil such as peanut oil, cotton seed oil, sesame oil, olive oil, corn oil and cacao fruit oil; glycols such as propylene glycol, glycerol, sorbitol, mannitol and polyethylene glycol; and polyol such as alginic acid when the composition is formulated as liquid. In this case, aqueous buffer including sodium salts, calcium salts, and optionally potassium salts can be used for injecting liquid pharmaceutical composition into bodies. Sodium salts, calcium salts and potassium salts may have halogenized type such as iodine or bromine, hydroxide, carbonate salt, hydrogen carbonate salt or sulfonate salts.

When the pharmaceutical composition is formulated as solid, the pharmaceutically acceptable carrier can comprise solid carrier such as solid filter, liquid filter or diluents, and encapsulating compound may be used as the carrier for administering the composition. For example, the pharmaceutically acceptable solid carrier can comprise, but is not limited to, sugar such as lactose, glucose and sucrose; starch such as corn starch of potato starch; cellulose or its derivative such as sodium carboxyl methyl cellulose, ethyl cellulose and cellulose acetate; powdered tragacanth; malt; gelatins; tallow; solid lubricant such as stearic acid and magnesium stearate; and calcium sulfate.

The pharmaceutically acceptable carrier can be selected as the administering types of the pharmaceutical composition of the present disclosure. In one embodiment, the pharmaceutical composition can be administered systemically. The administering route may comprise in oral, intracutaneous, intravenous, intra muscular, intra-articular, intrsynovial, intrathecal, intrahepatic, intralesional, intracranial, transdermal, intradermal, intrapumonal, intraperitoneal, intracardial, intraarterial, sublingual topical and/or intranasal.

Proper dosage of the pharmaceutical composition can be determined by common experiments using animal models. Such animal model includes a rabbit, a sheep, a mouse, a rat, a dog and primates other than humans. Proper injection unit dosage type includes sterilized aqueous solution, physiological saline solution and/or mixtures thereof. The solution may have adjusted pH of about 7.4.

The pharmaceutically acceptable carrier for injection may comprise hydrogel, adjusted release devices, delayed release devices, polylactic acid and collagen matrix. The pharmaceutically acceptable carrier appropriate for local uses may comprises lotion, cream, gel and similar thereof. If the composition is orally administered, tablet, capsule is preferred unit dosage form. The pharmaceutically acceptable carrier to be used for oral administering for the unit dosage form is well known in the art.

If necessary, the pharmaceutical composition may further comprise at least one auxiliary substance so as to further increase immunogenicity induced by the pharmaceutically active substance and/or the nucleic acid molecule. For example, such an auxiliary substance can include, but is not limited to, substances that allow maturation of dendritic cells (DCs), for example, lipopolysaccharides, TNF-alpha or CD40 ligand, GM-CSF and/or cytokines. More particularly, the auxiliary substance can include cytokines such as monokines, lymphokines, interleukins or chemokines that promotes the immune response such as various interleukins, interferons, GM-CSF, G-CSF, M-CSF, LT-beta or TNF-alpha, growth factors such as hGH.

The pharmaceutical composition can additionally include at least one additive such as buffering agents, stabilizers, surfactants, wetting agents, lubricants, emulsifiers, suspending agents, preservatives, antioxidants, opacifying agents, lubricants, processing aids, colorants, sweeteners, fragrances, flavoring agents, diluents, and other known additives that provide an attractive appearance to the drug (i.e., the nucleic acid molecule, gene carrier, or vaccine composition of the active ingredient of the present disclosure) or assist in the manufacture of pharmaceutical products (i.e., medicaments). As an example, the at least one additive can include emulsifiers such as Tween, wetting agents such as sodium lauryl sulfate, colorants, taste-imparting agents, agents forming tablets, stabilizers, antioxidants, and preservatives.

The contents of the nucleic acid molecule in the pharmaceutical composition are not particularly limited. In particular, when the RAN platform type nucleic acid molecule is contained in the composition, the nucleic acid molecule can be dissociated rapidly in body to secure safety and stability. In one embodiment, the contents of the nucleic acid molecule of the present disclosure in the pharmaceutical composition may be, but is not limited to, 1 to 1000 μg/ml, and preferably 10 to 1000 μg/ml.

An important factor for a suitable immune response is the stimulation of different T-cell sub-populations. T-lymphocytes typically differentiate into two sub-populations, the T-helper 1 (Th₁) cells and the T-helper 2 (Th₂) cells, with which the immune system is capable of destroying intracellular (Th₁) and extracellular (Th₂) pathogens (e.g., antigens). The two T-helper (Th) cell populations differ in the pattern of effector proteins (cytokines) produced by them. Thus, Th₁ cells assist the cellular immune response by activation of macrophages and cytotoxic T-cells. On the other hand, The cells promote the humoral immune response by stimulation of B-cells for conversion into plasma cells and by formation of antibodies (e.g. against antigens). The Th₁/Th₂ ratio is therefore of great importance in the immune response. In an exemplary embodiment, the nucleic acid molecule of the present disclosure can stimulate or enhance Th₁ immune response.

As an example, the pharmaceutical composition of the present disclosure can induce tumor-specific or pathogen-specific immune responses so that the composition can be used to prevent tumors and infectious diseases. Alternatively, the pharmaceutical composition can be used, but is not limited to, to prevent allergic disorders or diseases and/or auto-immune diseases.

The pharmaceutical composition of the present disclosure can be administered with any convenient dosage forms such as tablets, powders, capsules, solutions, dispersions, suspensions, syrups, sprays, suppositories, gels, emulsions, patches, and the likes. The composition can include any ingredients customary for pharmaceutical preparations, such as diluents, carriers, pH adjusters, sweeteners, bulking agents and additional active agents.

The pharmaceutical composition of the present disclosure can be prepared in unit dosage form by formulating the composition using the pharmaceutically acceptable carrier and/or recipient or by placing the composition in a multi-capacity container in accordance with a method that can be easily performed by a skilled person in the art to which the present disclosure pertains. In this case, the formulation can be in the form of a solution, a suspension or an emulsion in an oil or aqueous medium, or can be in the form of an extract, a powder, a granule, a tablet or a capsule. The formulation can additionally include a dispersant or a stabilizer.

On the other hand, the preparations herein may also contain more than one active compound, preferably compounds with complementary activities that do not adversely affect each other, if required for the particular indication to be treated. Alternatively or additionally, the pharmaceutical composition may include agents that enhance its function, such as, for example, cytotoxic agents, cytokines, chemotherapeutic agents, or growth-inhibiting or growth-enhancing agents. These molecules are suitably present in combination in amounts effective for the intended purpose.

In an alternative embodiment, the gene carrier including the nucleic acid molecule of the present disclosure can be included in the pharmaceutical composition. The gene carrier is designed to transfer and express the nucleotides encoding desired immunogens. The transcript of the gene of interest can be preferably present in a suitable expression construct to prepare the gene carrier. In the expression construct, the transcript of the gene of interest encoding the viral immunogens can be preferably linked operatively to the transcription control element TCCE.

For example, the expression construct can be the expression vector into which the above nucleic acid molecule is inserted. In this case, the vector can include the nucleic acid molecule, and the nucleic acid molecule can be linked to other nucleotides so that the nucleic acid molecule can encode fusion proteins or fusion peptides.

The methods of introducing the above gene carrier into cells can be carried out through various methods known in the art. In the present disclosure, when the gene carrier is manufactured based on the viral vector, the introduction can be carried out in accordance with a viral infection method known in the art. Alternatively or additionally, in case where the gene delivery system in the present disclosure is a naked DNA recombinant molecule or a plasmid, the genes can be transfected into cells by micro-injections, calcium phosphate precipitations, electroporation, liposome-mediated transfection, DEAE-dextran treatments, and gene bombardment methods.

### Example 1: Preparation of Nucleic Acid Molecule Having Inserted Encoding Influenza Virus Hemagglutinin (HA)

A nucleic acid molecule into which nucleotides encoding hemagglutinin (HA) of an influenza virus was inserted was prepared. The template DNA having a coding region encoding the HA of influenza virus from a nucleic acid molecule having MCS was designed as follows:

5′-KpnI recognition sequence (GGTACC)-T7 promoter (SEQ ID NO: 11)-first translation control element derived from human troponin 1 (TNNT1) (SEQ ID NO: 1)-PacI recognition sequence (TTAATTAA)-Kozak sequence (GCCACC)-Influenza virus HA (SEQ ID NO: 3) encoding sequence (HA, SEQ ID NO: 4)-ClaI recognition sequence (ATCGAT)-second translation control element derived from human troponin T1 (TNNT1) (SEQ ID NO: 2)-EcoRI recognition sequence (GAATTC)-polyadenylation signal (SEQ ID NO: 12)-SapI recognition sequence (GAAGAGC)-NotI recognition sequence (GCGGCCGC)-3′.

The template DNA was inserted into a pGH vector (downstream of SEQ ID NO: 13 and upstream of SEQD ID NO: 14), cloned and linearized by restriction endonuclease through in vitro transcription (IVT) so that the nucleic acid molecule of RNA platform type (hereinafter, “pHJ5L-HA”) was prepared.

### Experimental Example 1: In Vivo Mouse Immunization, Measurement of Antigen-Specific Immunoglobulins and Cytokines

Saline (Group 1) or PHJ5L-HA nucleic acid molecule (Group 2) was ministered into wild type Balb/c mouse. The RNA type nucleic acid molecule was purified by N-methyl pseudouridine (m1ψ) and cellulose and was formulated using lipid-based nano particles (LNP) used in preparation of COVID-19 mRNA vaccine of Moderna prior to administration.

The formulated pHJ5L-HA was injected intramuscularly into mice twice at two-week intervals, and two weeks later after the last immunization, the mice's blood was collected and organs were removed to confirm the induction of immunity by the nucleic acid molecule. The following Table 1 indicates the mouse immunization group in this Experimental Examples, and 5 mice were immunized per group.

TABLE 1
Mouse Immunization Group and Dosage Amount

| Group | N | Substrate | Dose | Route |
|---|---|---|---|---|
| Group 1 | 5 | Saline | 60 μl/mouse | Intramuscular |
| Group 2 | 5 | pHJ5L-HA + LNP | 60 μ/mouse | Intramuscular |

Two weeks later after the last immunization, the mice were euthanized with carbon dioxide, autopsied and mice blood was collected from the abdominal vein. The collected blood was left at room temperature for more than two hours to aggregate blood corpuscle, and the blood was centrifuges at 4000 g for 15 minutes to separate serum. After coating 100 ng of HA protein on a 96-well plate, blood was diluted 1:200 with 1% BSA in PBS as a primary antibody and 50 μl of the diluted blood was put into the well. After reacting at room temperature for 2 hours and washing tree times with 200 μl 0.5% PBST 200, 100 μl goat anti-mouse IgG H+I HRP conjugates as a secondary antibody diluted 1:3000 in 1% BSA in PBS was put into the well. After reacting for 1 hour at room temperature, the reactants were washed, and 100 μl TMA solution was added to check color development. When color development had progressed sufficiently, 50 μl of 2N sulfuric acid was added to terminate the reaction, and the absorbance was measured at a wavelength of 450 nm using a spectrometer that can measure the absorbance.

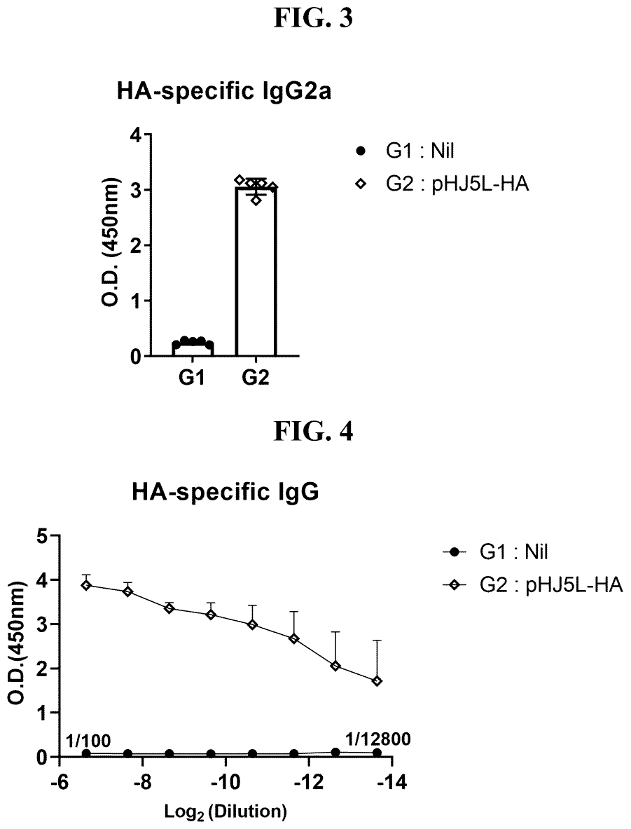

FIGS. 2, 3 and 4 indicate ELISA assay results measuring the amount of antigen-specific antibodies secreted from mice sera. Unlike negative control Group G1 injecting physiological saline, more antibodies specific to the HA protein were produced in the Group 2 immunized with the mRNA nucleic acid molecule prepared in Example 1. In particular, IgG1 relating to Th₂ immune response as well as IgG2a relating to Th1 immune response was produced in large quantities, and high absorbance was observed even when mice sera were diluted 12,800 times, which confirmed that much antibodies were produced.

### Experimental Example 2: Measurement of Neutralization Ability Through Hemagglutin Inhibition (HA) Assay

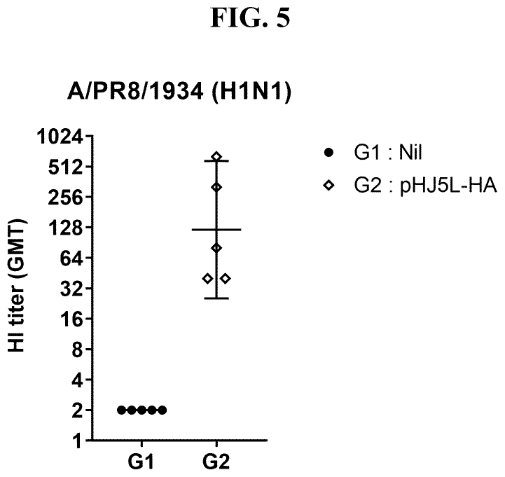

Mouse serum was obtained using the same method as in Experimental Example 1. 25 μl of PBS was added to the 96-well plate. 25 μl of mouse serum was put into the first column and sequentially diluted two-fold to the next column. 25 μl of Influenza virus PR8 HA, the same type of the HA encoded in the PHJ5L-HA mRNA, was put into each well and reacted for 30 minutes. 50 μl of 1% turkey blood was added and waited until red blood cells agglutinated. The reason red blood cells agglutinates is because of the HA antigen of the virus, and the reason red blood cells do not agglutinate is that there are HA antibodies in the serum. FIG. 5 illustrates the assay results. As illustrated in FIG. 5, it can be seen that the group G2 that put into pH5JL-HA mRNA had a high antibody titer by preventing red blood cells agglutination even at a high dilution rate.

### Experimental Example 3: Measurement of Neutralization Ability Through Microneutralization Assay

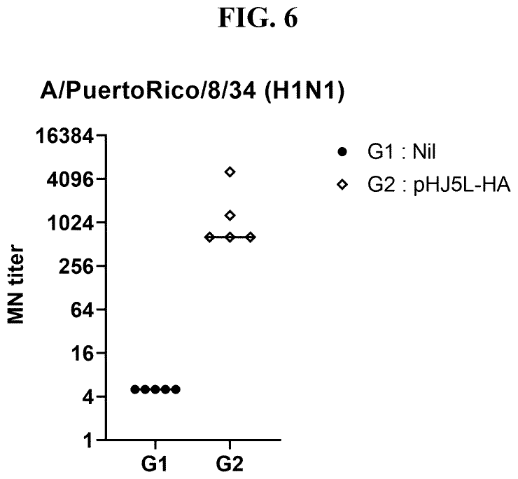

Mouse serum was obtained using the same method as in Experimental Example 1. MDCK cells (3×10⁴ cells/well) were spread in a 96-well plate. 12 μl of mouse serum was prepared and inactivated at 56° C. for 30 minutes. The inactivated serum was diluted 10 times with medium and 80 μl of the diluted serum was put into the first column. In other column, 40 μl of medium was put into, and the serum of the first column was diluted 2-fold, exceeding 40 μl each. 40 μl of virus PR8 HA at a concentration of TCID50/40 μl was put into each column, and reacted for 1 hour in an incubator at 37° C. 50 μl of the reacted virus and serum were put into on MDCK cells in the 96-well plate from which the medium had been removed, and the cells were infected in a 37° C. incubator for 2 hours. After 2 hours, 50 μl of complete medium was added to each well and placed in an incubator at 37° C. Cells were observed over time, and when cell lesions occurred, the whole supernatant was removed, 4% formaldehyde solution was added, and the cells were fixed for more than 3 hours. 2% crystal violet solution was added to each well for staining for over 30 minutes, washed the solution, and virus titer was measured. FIG. 6 illustrates the assay results. It was confirmed that the group G2 immunized with pHJ5L-HA mRNA effectively neutralized the virus.

### Experimental Example 4: Measurement of Antigen-Specific IFN-Gamma

### (1) ELISPOT Assay

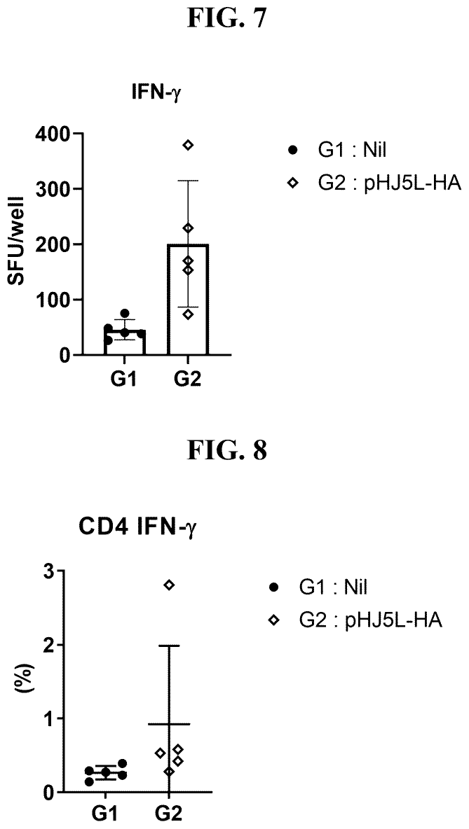

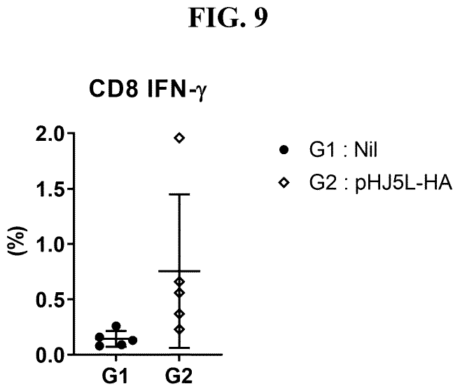

Mouse spleen extracted in sacrificing mice immunized by the immunization schedule in Experimental Example 1 was ground using a 40 μm strainer to produce as many single cells as possible. 50 μl of the ground spleen cells were put into a pre-coating ELISpot plate at a number of 5×10⁵ cells/well. For the non-stimulation group G1, 50 μl of RPMI1640 complete medium (10% FBS, 1% antibiotics) was added. For the peptide stimulation group G2 expressed from the pHJ5L-HA mRNA, 50 μl of 7 types of HA peptides were added to the complete medium at a concentration of 4 μl/well. After incubating in an incubator at 37° C. for 48 hours, the experiment was performed by the protocol provided by the manufacturer. FIG. 7 illustrates the measurement result. Compared to the negative control group G1, the number of spots increased in the group G2 immunized with pHJ5L-HA mRNA, confirming that the number of cells secreting IFN-gamma increased significantly.

### (2) Flow Cytometry Using FACS

The spleen cells grounded above were put into a round bottom 96-well plate at a number of 1×10⁶ cells/well, then stimulated by adding 7 types of HA peptides at a concentration of 4 μg/well, and cultured in an incubator at 37° C. for 24 hours. After staining the cultured cells with a T-cell specific antibody and IFN-gamma specific antibody, T cells producing IFN-gamma were distinguished through flow cytometry. FIGS. 8 and 9 illustrate the assay results. Compared to the negative control group G1, the number of T-cells secreting IFN-gamma significantly increased in the group immunized with the mRNA G2.

### Example 2: Preparation of Nucleic Acid Molecule Having Inserted Encoding SFTSF Glycoprotein N (Gn)

A nucleic acid molecule of an RAN platform type (hereinafter, referred to as “pHJ5L-Gn”) was prepared by repeating the procedure of Example 1 except that a modified nucleotide (SEQ ID NO: 6) encoding glycoprotein N (SEQ ID NO: 5) of SFTSV was inserted in the coding region instead of the nucleotide encoding the influenza virus HA.

### Example 3: Preparation of Nucleic Acid Molecule Having Inserted Encoding Deleting SFTSF Glycoprotein N (Gn)

A nucleic acid molecule of an RAN platform type (hereinafter, referred to as “pHJ5L-GnΔTM”) was prepared by repeating the procedure of Example 1 except that a modified nucleotide (SEQ ID NO: 8) encoding deleted glycoprotein N (SEQ ID NO: 7) of SFTSV was inserted in the coding region instead of the nucleotide encoding the influenza virus HA.

### Example 3: Preparation of Nucleic Acid Molecule Having Inserted Encoding Deleting SFTSF Glycoprotein N (Gn)

A nucleic acid molecule of an RAN platform type (hereinafter, referred to as “pHJ5L-GnΔSTEM”) was prepared by repeating the procedure of Example 1 except that a modified nucleotide (SEQ ID NO: 10) encoding deleted glycoprotein N (SEQ ID NO: 9) of SFTSV was inserted in the coding region instead of the nucleotide encoding the influenza virus HA.

### Experimental Example 5: Certification of Expression of Nucleic Acid Molecules

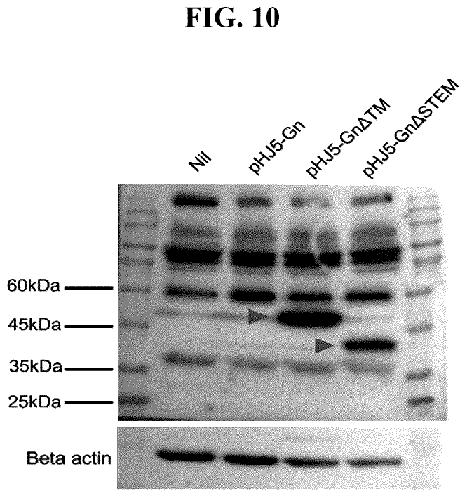

Expressions of the target peptides from the mRNA prepared in Examples 2 to 4 was confirmed. VERO cells (6×10⁵) were inoculated into a 6-well cell culture plate with 10% FBS DMEM complete medium and incubated O/N at 37° C. 10 μg of the nucleic acid molecules were transfected with Lipofectamine2000™ and incubated at 37° C. for 30 hours. After incubation, intracellular proteins were quantified and SDA PAGE was performed on 30 μg of the nucleic acid molecules. The expression of Gn, GnΔTM and GnΔSTEM proteins were confirmed by Western blot. SFTS Virus HB29 Antibody (NBP2-41153) as a primary antibody was diluted 1:1000 in 5% skim milk in PBST and incubated O/N at 4° C., and anti-rabbit IgG HRP antibody as a secondary antibody was diluted 1:5000 in 5% skim milk in PBSS and incubated at room temperature for 1 hour. As illustrated in FIG. 10, a protein of about 60 kDa was expressed in the pHJ5L-GN nucleic acid molecule, a protein of about 50 kDa was expressed in the pHJ5L-ΔTM nucleic acid molecule, and a protein of about 40 kDa was expressed in the pHJ5L-ΔSTTM nucleic acid molecule.

### Experimental Example 6: In Vivo Mouse Immunization, Measurement of Antigen-Specific Immunoglobulins

Saline (Group 1) or PHJ5L-Gn nucleic acid molecule (Group 2) was ministered into wild type Balb/c mouse. Other than that, mice were injected intramuscularly with the same immunization schedule and dose as in Experimental Example 1, and then blood was collected and organs were removed from the mice 2 weeks later after the last immunization to confirm whether immunity was induced by the mRNA. The mouse groups used in this Experimental Example were the same as Table 1 in Experimental Example 1, and 5 mice per groups were immunized.

Two weeks later after the last immunization, the mice were euthanized with carbon dioxide, autopsied and mice blood was collected from the abdominal vein. The collected blood was left at room temperature for more than two hours to aggregate blood corpuscle, and the blood was centrifuges at 4000 g for 15 minutes to separate serum. After coating 100 ng of Gn protein on a 96-well plate, the amount of antigen-specific antibody secreted in the mouse serum was measured by EILSA assay in the same manner as in Experimental Example 1. FIGS. 11 and 12 illustrate the measurement results. Unlike negative control Group G1 injecting physiological saline, much antibodies specific to the Gn protein were produced in the Group 2 immunized with the pHJ5L-Gn mRNA. In particular, IgG1 relating to The immune response as well as IgG2a relating to Th1 immune response was produced in large quantities.

### Experimental Example 7: Measurement of Antigen-Specific IFN-Gamma

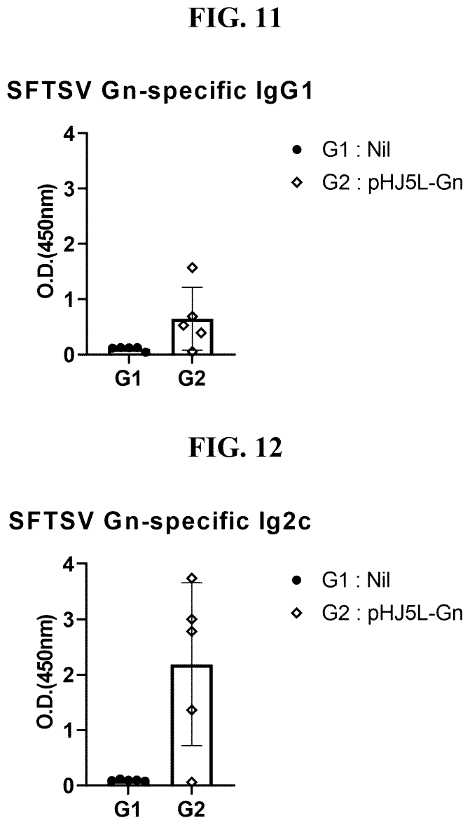

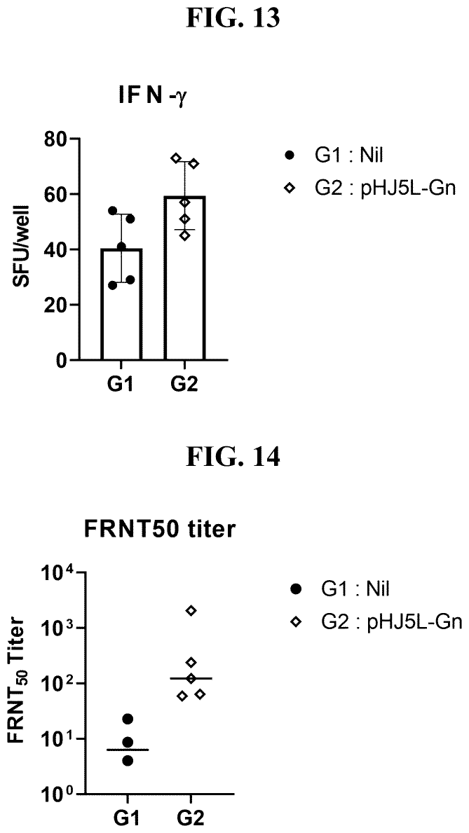

Mouse spleen extracted in sacrificing mice immunized by the immunization schedule in Experimental Example 6 was ground using a 40 μm strainer to produce as many single cells as possible. For the peptides stimulation group G2 expressed in pHJ5L-Gn mRNA, Gn protein was added to the complete medium at a concentration of 200 ng/well and 50 μl of the Gn-added medium was put into the well. After incubating in an incubator at 37° C. for 48 hours, the experiment was performed by the protocol provided by the manufacturer. FIG. 13 illustrates the measurement result. Compared to the negative control group G1, the number of spots increased in the group G2 immunized with pHJ5L-Gn mRNA, confirming that the number of cells secreting IFN-gamma increased significantly.

### Experimental Example 8: Measurement of Neutralization Ability

Vero cells (1.5×10⁵ cells/well) were inoculated into a 24-well plate and cultured in an incubator at 37° C. 60 μl of serum obtained from mice was diluted 1/10 in 540 μl of medium and inactivated at 56° C. for 30 minutes. The inactivated serum was diluted by 1/2 by adding 300 μl 300 μl of medium in advance, and the initially diluted 10-fold serum was diluted up to 320-fold. 200 μl of diluted serum and 200 μl of SFTS virus solution at a concentration of 80 ffu/well were mixed. After removing the complete medium from the pre-inoculated cells and washing them with PBS, 100 μl of the solution mixed with serum and virus was added to each well, and reacted in an incubator at 37° C. for 1 hour. At this time, shake it every 20 minutes. After 1 hour, the virus solution was removed, and 1 ml of 1.5% carbosyl-methylcellulose solution was added to each well and left in an incubator at 37°° C. for 2 days. After 2 days, the supernatant was removed, 4% formaldehyde solution was added, and the virus was inactivated by leaving it at room temperature for 10 minutes. After 10 minutes, the supernatant was removed and washed with PBS. Diluted 1:500 in PBS, 500 μl of anti-SFTSV NP monoclonal antibody in 0.5% Triton The supernatant was removed and washed with PBS. As a secondary antibody, HRP-conjugated antibody containing 0.5% Triton. The supernatant was removed again and washed with PBS. Afterwards, add 500 μl of DAB substrate to each well and react until the foci turn brown, then wash with tap water to stop the reaction. At this time, the dilution factor at which the number of stained dots is half the number of wells containing only virus without serum is called FRNT50. A higher dilution factor means more neutralizing antibodies. FIG. 14 illustrates the measurement result. It can be confirmed that a high level of neutralizing antibodies exists in the serum of the group immunized with mRNA.

While the present disclosure has been described with reference to exemplary embodiments and examples, the present disclosure is not limited to those embodiments and examples. Rather, it will be apparent to those skilled in the art that various modifications and variations can be made in the present disclosure without departing from the scope of the disclosure. However, it is apparent that such modifications and variations are within the scope of the present disclosure from the appended claims.

What is claimed is:

1. A nucleic acid molecule comprising:
a translation control element derived from troponin T1 (TNNT1); and
a coding region linked operatively to the translation control element,
wherein the coding region includes a nucleotide encoding an immunogen of an influenza virus or a severe fever with thrombocytopenia syndrome virus (SFTSV) or fragments thereof.

2. The nucleic acid molecule of claim 1, wherein the translation control element comprises a first translation control element upstream of the coding region and a second translation control element downstream of the coding region.

3. The nucleic acid molecule of claim 2, wherein the first translation control element comprises a nucleotide of SEQ ID NO: 1 or a transcript thereof.

4. The nucleic acid molecule of claim 2, the second translation control element comprises a nucleotide of SEQ ID NO: 2 or a transcript thereof.

5. The nucleic acid molecule of claim 1, wherein the immunogen of the influenza virus comprises hemagglutinin of the influenza virus or fragments thereof.

6. The nucleic acid molecule of claim 1, wherein the immunogen of the influenza virus comprises an amino acid of SEQ ID NO: 3.

7. The nucleic acid molecule of claim 1, wherein the nucleotide encoding the immunogen of the influenza virus or fragments thereof comprises a nucleotide of SEQ ID NO: 4 or a transcript thereof.

8. The nucleic acid molecule of claim 1, wherein the immunogen of the severe fever with thrombocytopenia syndrome virus comprises a glycoprotein N of the sever fever with thrombocytopenia syndrome virus or fragments thereof.

9. The nucleic acid molecule of claim 1, wherein the immunogen of the severe fever with thrombocytopenia syndrome virus comprises an amino acid selected from SEQ ID NO: 5, SEQ ID NO: 7 and SEQ ID NO: 9.

10. The nucleic acid molecule of claim 1, wherein the nucleotide encoding the immunogen of the severe fever with thrombocytopenia syndrome virus or fragments thereof comprises a nucleotide selected from SEQ ID NO: 6, SEQ ID NO: 8 and SEQ ID NO: 10 or transcripts thereof.

11. The nucleic acid molecule of claim 1, wherein the nucleic acid molecule comprises an RNA type nucleic acid molecule.

12. The nucleic acid molecule of claim 1, wherein the nucleic acid molecule further comprises at least one of the following nucleotides:
a transcription control element linked operatively to the coding region; and
a polyadenylation signal sequence or a poly adenosine sequence downstream of the transcription control element.

13. A recombination expression vector comprising a nucleic acid molecule, the nucleic acid molecule comprises:
a translation control element derived from troponin T1 (TNNT1); and
a coding region linked operatively to the translation control element,
wherein the coding region includes a nucleotide encoding an immunogen of an influenza virus or a severe fever with thrombocytopenia syndrome virus (SFTSV) or fragments thereof.

14. The recombination expression vector of claim 13, wherein the translation control element comprises a first translation control element upstream of the coding region and a second translation control element downstream of the coding region.

15. The recombination expression vector of claim 14, wherein the first translation control element comprises a nucleotide of SEQ ID NO: 1 or a transcript thereof.

16. The recombination expression vector of claim 15, the second translation control element comprises a nucleotide of SEQ ID NO: 2 or a transcript thereof.

17. The recombination expression vector of claim 13, wherein the immunogen of the influenza virus comprises hemagglutinin of the influenza virus or fragments thereof.

18. The recombination expression vector of claim 13, wherein the immunogen of the influenza virus comprises an amino acid of SEQ ID NO: 3.

19. The recombination expression vector of claim 13, wherein the nucleotide encoding the immunogen of the influenza virus or fragments thereof comprises a nucleotide of SEQ ID NO: 4 or a transcript thereof.

20. The recombination expression vector of claim 13, wherein the immunogen of the severe fever with thrombocytopenia syndrome virus comprises a glycoprotein N of the sever fever with thrombocytopenia syndrome virus or fragments thereof.

21. The recombination expression vector of claim 13, wherein the immunogen of the severe fever with thrombocytopenia syndrome virus comprises an amino acid selected from SEQ ID NO: 5, SEQ ID NO: 7 and SEQ ID NO: 9.

22. The recombination expression vector of claim 13, wherein the nucleotide encoding the immunogen of the severe fever with thrombocytopenia syndrome virus or fragments thereof comprises a nucleotide selected from SEQ ID NO: 6, SEQ ID NO: 8 and SEQ ID NO: 10 or transcripts thereof.

23. The recombination expression vector of claim 13, wherein the nucleic acid molecule comprises an RNA type nucleic acid molecule.

24. The recombination expression vector of claim 13, wherein the nucleic acid molecule further comprises at least one of the following nucleotides:
a transcription control element linked operatively to the coding region; and
a polyadenylation signal sequence or a poly adenosine sequence downstream of the transcription control element.

25. A pharmaceutical composition for treating or preventing influenza or a severe fever with thrombocytopenia syndrome comprising the nucleic acid molecule of claim 1 or an expression construct in which the nucleic acid molecule of claim 1 is inserted.

26. The pharmaceutical composition of claim 25, wherein the pharmaceutical composition further comprises at least one of an adjuvant, a nucleic acid stabilizer and a lipid nano particle.


SEQUENCE LISTING (14 sequences)

SEQ ID NO: 1 - DNA, 57 bp, Homo sapiens
  source 1..57: mol_type = genomic DNA
agcaaggctcagcctcaagattcacagcatctcagacacagcctaggccgcaccagg

SEQ ID NO: 2 - DNA, 217 bp, Homo sapiens
  source 1..217: mol_type = genomic DNA
ggatgccgccccggacagtggcacctgggaagcctgggagtgtttgtcccatcggtagcttgaaataaacgctcccctcagacacccgctgggttctctgatgttattatggttgagatgcagctggtctctcctggttaattgacttcttattaccaacaagttacttgaaagagtgtcaccaataatcattaaagtacggcaggctgaaccttca

SEQ ID NO: 3 - protein, 565 aa, Influenza virus
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 4 - DNA, 1698 bp, Influenza virus
  source 1..1698: mol_type = genomic DNA
atgaaggccaacctgctcgtgctgctgtgcgccctcgcggccgccgacgccgacaccatctgcatcggctaccacgccaacaacagcaccgacacggtcgacaccgtgctggagaagaacgtgaccgtcacccactccgtgaacctgctcgaggacagccacaacgggaagctgtgccggctgaagggcatcgcgcccctccagctggggaagtgcaacatcgccggctggctgctcgggaacccggagtgcgaccccctgctgcccgtgcgctcctggagctacatcgtcgagacgcccaactccgagaacggcatctgctacccgggcgacttcatcgactacgaggagctccgggagcagctgagctccgtgagctccttcgagcgcttcgagatcttccccaaggagagctcctggcccaaccacaacaccaacggggtgaccgccgcctgcagccacgagggcaagtccagcttctaccggaacctgctctggctgaccgagaaggaggggtcctaccccaagctgaagaacagctacgtcaacaagaagggcaaggaggtgctcgtgctgtgggggatccaccacccgcccaactccaaggagcagcagaacctgtaccagaacgagaacgcgtacgtcagcgtggtgacgtccaactacaaccgccggttcacccccgagatcgccgagcgccccaaggtccgggaccaggccggccgcatgaactactactggaccctcctgaagccgggcgacaccatcatcttcgaggccaacgggaacctgatcgccccgatgtacgcgttcgccctcagccggggcttcgggagcggcatcatcacgtccaacgccagcatgcacgagtgcaacaccaagtgccagacccccctgggcgccatcaactccagcctgccctaccagaacatccacccggtgaccatcggggagtgccccaagtacgtgcgctccgccaagctccggatggtcacgggcctgcgcaacaaccccagcatccagtcccgggggctgttcggcgcgatcgccgggttcatcgagggcggctggaccgggatgatcgacggctggtacgggtaccaccaccagaacgagcagggcagcgggtacgccgccgaccagaagtccacccagaacgccatcaacggcatcaccaacaaggtgaacacggtgatcgagaagatgaacatccagttcaccgcggtcggcaaggagttcaacaagctcgagaagcgcatggagaacctgaacaagaaggtggacgacgggttcctggacatctggacctacaacgccgagctcctggtgctgctcgagaacgagcggaccctggacttccacgacagcaacgtcaagaacctgtacgagaaggtgaagtcccagctcaagaacaacgccaaggagatcggcaacgggtgcttcgagttctaccacaagtgcgacaacgagtgcatggagagcgtccgcaacggcacgtacgactaccccaagtactccgaggagagcaagctgaaccgggagaaggtggacggggtgaagctggagtccatgggcatctaccagatcctcgccatctacagcaccgtcgcctccagcctggtgctgctggtgtccctcggcgcgatcagcttctggatgtgcagcaacgggtccctgcagtgccgcatctgcatctga

SEQ ID NO: 5 - protein, 1073 aa, synthetic construct
MMKVIWFSSLICLVIQCSGDSGPIICAGPIHSNKSASIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKACKTSGSCRGDMQFCKVAGCEHGEEASDAKCRCSLVHKPGEVVVSYGGMRVRPKCYGFSRMMATLEVNPPEQRIGQCTGCHLECINGGVRLITLTSELKSATVCASHFCSSATSGKKSTEIQFHSGSLVGKTAIHVKGALVDGTEFTFEGSCMFPDGCDAVDCTFCREFLKNPQCYPAKKWLFILIVILLGYAGLMLITNVLKAIGVWGSWVIAPVKLMFAIIKKLMRTVSCLMGKLMDRGRQVIHEEIGENGEGNQDDVRIEMARPRRVRHWMYSPVILTILAIGLAEGCDEMVHADSKLVSCRQGSGNMKECVTTGRALLPAVNPGQEACLHFTAPGSPDSKCLKIKVKRINLKCKKSSSYFVPDARSRCTSVRRCRWAGDCQSGCPPHFTSNSFSDDWAGKMDRAGLGFSGCSDGCGGAACGCFNAAPSCIFWRKWVENPHGIIWKVSPCAAWVPSAVIELTMPSGEVRTFHPMSGIPTQVFKGVSVTYLGSDMEVSGLTDLCEIEELKSKKLALAPCNQAGMGVVGKVGEIQCSSEESARTIKKDGCIWNADLVGIELRVDDAVCYSKITSVEAVANYSAIPTTIGGLRFERSHDSQGKISGSPLDITAIRGSFSVNYRGLRLSLSEITATCTGEVTNVSGCYSCMTGAKVSIKLHSSKNSTAHVRCKGDETAFSVLEGVHSYTVSLSFDHAVVDEQCQLNCGGHESQVTLKGNLIFLDVPKFVDGSYMQTYHSTVPTGANIPSPTDWLNALFGNGLSRWILGVIGVLLGGLALFFLIMSLFKLGTKQVFRSRTKLA

SEQ ID NO: 6 - DNA, 3225 bp, synthetic construct
  source 1..3225: mol_type = other DNA
atgatgaaagtgatttggttctcctctctgatttgtctggtcattcagtgtagcggggattctggacctattatctgtgctgggccaatccacagcaacaagagcgcctccatcccccacctgctgggctactccgagaagatctgccagatcgaccgcctgatccacgtgagctcctggctgcggaaccacagccagttccagggatacgtgggacagaggggaggccgcagccaggtgtcctactatccagccgagaattcttatagcagatggtccggcctgctgtctccatgtgacgcagattggctgggcatgctggtggtgaagaaggccaagggctctgatatgatcgtgcctggcccaagctacaagggcaaggtgttctttgagcggcccaccttcgacggatatgtgggatggggatgcggatctggcaagagcaggacagagtccggcgagctgtgcagcagcgattctggcacctcctctggcctgctgcctagcgatcgcgtgctgtggatcggcgacgtggcatgccagccaatgacacccatccctgaggagacattcctggagctgaagtccttctctcagagcgagtttcctgatatctgcaagatcgacggcatcgtgttcaatcagtgtgagggcgagagcctgccacagccctttgatgtggcctggatggacgtgggccactcccacaagatcatcatgcgggagcacaagaccaagtgggtgcaggagagctcctctaaggacttcgtgtgctacaaggagggcacaggcccatgttccgagtctgaggagaaggcctgcaagaccagcggctcctgtagaggcgatatgcagttttgcaaggtggcaggatgtgagcacggagaggaggcctctgacgccaagtgcaggtgtagcctggtgcacaagccaggagaggtggtggtgtcttacggaggaatgcgggtgcggcccaagtgctatggcttcagcagaatgatggccacactggaggtgaacccccctgagcagaggatcggccagtgcaccggctgtcacctggagtgtatcaatggcggcgtgaggctgatcaccctgacaagcgagctgaagtccgccacagtgtgcgccagccacttctgtagctccgccacatctggcaagaagagcaccgagatccagtttcactctggcagcctggtgggcaagaccgcaatccacgtgaagggcgccctggtggatggcacagagttcacctttgagggctcctgcatgttcccagacggctgtgatgccgtggactgcaccttctgtagagagtttctgaagaacccacagtgctaccccgccaagaagtggctgtttatcctgatcgtgatcctgctgggctatgccggcctgatgctgatcacaaatgtgctgaaggccatcggcgtgtggggatcttgggtcatcgcacccgtgaagctgatgtttgccatcatcaagaagctgatgaggaccgtgtcctgcctgatgggcaagctgatggacaggggccggcaggtcatccacgaggagatcggcgagaacggcgagggcaatcaggacgatgtgcggatcgagatggccagacctcggagagtgaggcactggatgtacagcccagtgatcctgacaatcctggcaatcggcctggcagagggatgcgatgagatggtgcacgccgactccaagctggtgtcttgccgccagggctccggcaacatgaaggagtgcgtgaccacaggccgggccctgctgcctgcagtgaatccaggccaggaggcatgtctgcacttcaccgcaccaggctcccctgactctaagtgcctgaagatcaaggtgaagcgcatcaacctgaagtgtaagaagtctagctcctattttgtgcccgatgcccggagcagatgcacatccgtgaggcgctgtagatgggcaggcgactgccagagcggctgtccaccccacttcacctccaattcttttagcgacgattgggccggcaagatggatagagcaggcctgggcttcagcggatgctccgacggatgtggaggagcagcatgcggatgtttcaacgcagccccctcctgcatcttttggaggaagtgggtggagaatcctcacggcatcatctggaaggtgagcccatgtgcagcatgggtgccctccgccgtgatcgagctgacaatgccatccggagaggtgcgcaccttccaccccatgtctggcatccctacacaggtgtttaagggcgtgagcgtgacctacctgggcagcgatatggaggtgtccggcctgaccgacctgtgcgagatcgaggagctgaagtccaagaagctggccctggcaccttgtaaccaggcaggaatgggagtggtgggcaaggtcggcgagatccagtgctctagcgaggagagcgcccgcacaatcaagaaggatggctgtatctggaacgcagacctggtgggaatcgagctgagggtggacgatgccgtgtgctactctaagatcaccagcgtggaggccgtggccaattattccgccatccctaccacaatcggcggcctgcggttcgagagatctcacgatagccagggcaagatctccggctctccactggacatcaccgccatccgcggcagcttttccgtgaactacaggggcctgcgcctgtctctgagcgagatcaccgccacatgcaccggcgaggtgacaaacgtgagcggctgctattcttgtatgaccggcgccaaggtgtctatcaagctgcactcctctaagaacagcacagcccacgtgcggtgtaagggcgatgagacagccttctccgtgctggagggcgtgcactcttatacagtgtccctgtcttttgatcacgccgtggtggacgagcagtgccagctgaactgtggcggccacgagagccaggtgaccctgaagggcaatctgatcttcctggatgtgccaaagtttgtggacggcagctacatgcagacatatcactccacagtgcccaccggcgccaatatcccttccccaaccgactggctgaacgccctgttcggcaatggcctgtctaggtggatcctgggcgtgatcggcgtgctgctgggaggcctggccctgttctttctgatcatgtctctgttcaaactgggcactaagcaggtctttcggtcccgcacaaaactggcttaatga

SEQ ID NO: 7 - protein, 452 aa, unidentified
MMKVIWFSSLICLVIQCSGDSGPIICAGPIHSNKSASIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKACKTSGSCRGDMQFCKVAGCEHGEEASDAKCRCSLVHKPGEVVVSYGGMRVRPKCYGFSRMMATLEVNPPEQRIGQCTGCHLECINGGVRLITLTSELKSATVCASHFCSSATSGKKSTEIQFHSGSLVGKTAIHVKGALVDGTEFTFEGSCMFPDGCDAVDCTFCREFLKNPQCYPAKK

SEQ ID NO: 8 - DNA, 1359 bp, synthetic construct
  source 1..1359: mol_type = other DNA
atgatgaaagtgatttggttctcctctctgatttgtctggtcattcagtgtagcggggattctggacctattatctgtgctgggccaatccacagcaacaagagcgcctccatcccccacctgctgggctactccgagaagatctgccagatcgaccgcctgatccacgtgagctcctggctgcggaaccacagccagttccagggatacgtgggacagaggggaggccgcagccaggtgtcctactatccagccgagaattcttatagcagatggtccggcctgctgtctccatgtgacgcagattggctgggcatgctggtggtgaagaaggccaagggctctgatatgatcgtgcctggcccaagctacaagggcaaggtgttctttgagcggcccaccttcgacggatatgtgggatggggatgcggatctggcaagagcaggacagagtccggcgagctgtgcagcagcgattctggcacctcctctggcctgctgcctagcgatcgcgtgctgtggatcggcgacgtggcatgccagccaatgacacccatccctgaggagacattcctggagctgaagtccttctctcagagcgagtttcctgatatctgcaagatcgacggcatcgtgttcaatcagtgtgagggcgagagcctgccacagccctttgatgtggcctggatggacgtgggccactcccacaagatcatcatgcgggagcacaagaccaagtgggtgcaggagagctcctctaaggacttcgtgtgctacaaggagggcacaggcccatgttccgagtctgaggagaaggcctgcaagaccagcggctcctgtagaggcgatatgcagttttgcaaggtggcaggatgtgagcacggagaggaggcctctgacgccaagtgcaggtgtagcctggtgcacaagccaggagaggtggtggtgtcttacggaggaatgcgggtgcggcccaagtgctatggcttcagcagaatgatggccacactggaggtgaacccccctgagcagaggatcggccagtgcaccggctgtcacctggagtgtatcaatggcggcgtgaggctgatcaccctgacaagcgagctgaagtccgccacagtgtgcgccagccacttctgtagctccgccacatctggcaagaagagcaccgagatccagtttcactctggcagcctggtgggcaagaccgcaatccacgtgaagggcgccctggtggatggcacagagttcacctttgagggctcctgcatgttcccagacggctgtgatgccgtggactgcaccttctgtagagagtttctgaagaacccacagtgctaccccgccaagaagtga

SEQ ID NO: 9 - protein, 339 aa, synthetic construct
MMKVIWFSSLICLVIQCSGDSGPIICAGPIHSNKSASIPHLLGYSEKICQIDRLIHVSSWLRNHSQFQGYVGQRGGRSQVSYYPAENSYSRWSGLLSPCDADWLGMLVVKKAKGSDMIVPGPSYKGKVFFERPTFDGYVGWGCGSGKSRTESGELCSSDSGTSSGLLPSDRVLWIGDVACQPMTPIPEETFLELKSFSQSEFPDICKIDGIVFNQCEGESLPQPFDVAWMDVGHSHKIIMREHKTKWVQESSSKDFVCYKEGTGPCSESEEKACKTSGSCRGDMQFCKVAGCEHGEEASDAKCRCSLVHKPGEVVVSYGGMRVRPKCYGFSRMMATLEV

SEQ ID NO: 10 - DNA, 1020 bp, synthetic construct
  source 1..1020: mol_type = other DNA
atgatgaaagtgatttggttctcctctctgatttgtctggtcattcagtgtagcggggattctggacctattatctgtgctgggccaatccacagcaacaagagcgcctccatcccccacctgctgggctactccgagaagatctgccagatcgaccgcctgatccacgtgagctcctggctgcggaaccacagccagttccagggatacgtgggacagaggggaggccgcagccaggtgtcctactatccagccgagaattcttatagcagatggtccggcctgctgtctccatgtgacgcagattggctgggcatgctggtggtgaagaaggccaagggctctgatatgatcgtgcctggcccaagctacaagggcaaggtgttctttgagcggcccaccttcgacggatatgtgggatggggatgcggatctggcaagagcaggacagagtccggcgagctgtgcagcagcgattctggcacctcctctggcctgctgcctagcgatcgcgtgctgtggatcggcgacgtggcatgccagccaatgacacccatccctgaggagacattcctggagctgaagtccttctctcagagcgagtttcctgatatctgcaagatcgacggcatcgtgttcaatcagtgtgagggcgagagcctgccacagccctttgatgtggcctggatggacgtgggccactcccacaagatcatcatgcgggagcacaagaccaagtgggtgcaggagagctcctctaaggacttcgtgtgctacaaggagggcacaggcccatgttccgagtctgaggagaaggcctgcaagaccagcggctcctgtagaggcgatatgcagttttgcaaggtggcaggatgtgagcacggagaggaggcctctgacgccaagtgcaggtgtagcctggtgcacaagccaggagaggtggtggtgtcttacggaggaatgcgggtgcggcccaagtgctatggcttcagcagaatgatggccacactggaggtgtga

SEQ ID NO: 11 - DNA, 19 bp, Bacteriophage T7
  source 1..19: mol_type = genomic DNA
taatacgactcactatagg

SEQ ID NO: 12 - DNA, 110 bp, synthetic construct
  source 1..110: mol_type = other DNA
aaaaaaaaaaaaaaaaaaaaaaaaaaaaaaaaaaaaaaaaaaaaaaaaaagatcatcagtaaaaaaaaaaaaaaaaaaaaaaaaaaaaaaaaaaaaaaaaaaaaaaaaaa

SEQ ID NO: 13 - DNA, 2223 bp, synthetic construct
  source 1..2223: mol_type = other DNA
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggatctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagcttgcaagcttcacgtgat

SEQ ID NO: 14 - DNA, 684 bp, synthetic construct
  source 1..684: mol_type = other DNA
atcccgggaagcttggccttgacggccgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag